(19) United States
(12) Patent Application Publication
(10) Pub. No.: US 2026/0151540 A1
(43) Pub. Date: Jun. 4, 2026

(54) Method for Preparing a Sterile Hydrogel Comprising a Cross-Linked or Non-Crosslinked Polysaccharide or a Mixture Thereof

(71) Applicant: TEOXANE SA, GENEVE (CH)

(72) Inventors: Romain BRUSINI, VALLEIRY (FR); Camille VANTOU, ECHENEVEX (FR); Killian FLEGEAU, NYON (CH); Laura Jing JING, ST JULIEN EN GENEVOIS (FR); Jimmy FAIVRE, VALSERHONE (FR); François BOURDON, GAILLARD (FR)

(21) Appl. No.: 19/164,454

(22) PCT Filed: Mar. 21, 2024
(86) PCT No.: PCT/EP2024/057664

§ 371 Date: Sep. 11, 2025

(30) Foreign Application Priority Data
Mar. 21, 2023 (FR) FR2302637

(51) Int. Cl. A61L 27/52; A61L 27/20; A61L 27/54; C08J 3/075; C08J 3/24

(52) U.S. Cl. CPC A61L 27/52; A61L 27/20; A61L 27/54; C08J 3/075; C08J 3/246; A61L 2300/402; A61L 2430/34; C08J 2305/08

(57) ABSTRACT

The present invention concerns a sterile hydrogel comprising a cross-linked polysaccharide, a non-cross-linked polysaccharide or a mixture thereof, in particular comprising a cross-linked hyaluronic acid, a non-cross-linked hyaluronic acid or a mixture thereof, and further comprising zinc and citrate ions, as well as a method for preparing same.

### FIELD OF THE INVENTION

The present invention concerns a sterile hydrogel comprising a cross-linked polysaccharide, a non-cross-linked polysaccharide or a mixture thereof, in particular comprising a cross-linked hyaluronic acid, a non-cross-linked hyaluronic acid or a mixture thereof, and further comprising zinc and citrate ions, as well as a method for preparing same.

### TECHNOLOGICAL BACKGROUND

Polysaccharides, such as glycosaminoglycans, are widely used in the medical and esthetic fields, especially for filling soft tissues. In particular, the majority of products marketed for esthetic applications are based on hyaluronic acid. To improve skin quality, hydrogels prepared from unmodified hyaluronic acid are interesting because they have the advantage of being perfectly biocompatible.

It is also possible to use hydrogels based on modified hyaluronic acid, hyaluronic acid being usually modified by cross-linking. This cross-linking has the advantage of increasing the in vivo durability and in vivo resistance to degradation of hydrogels. Hydrogels based on cross-linked hyaluronic acid can be obtained by various preparation methods.

Currently, there is an increasing need to provide hydrogels with an improved biocompatibility profile capable of delivering beneficial biological effects for skin quality. In this context, zinc proves to be an element of choice. It is a micronutrient that has many beneficial biological effects such as being a cofactor of many enzymes, especially those involved in the healing and reconstruction processes of the extracellular matrix. In addition to healing activities, anti-inflammatory and anti-infective activities have also been associated with zinc. Zinc may therefore be interesting to reduce any side effects due to the inflammatory response associated with hydrogel administration.

However, the preparation of hydrogels comprising zinc is not easy, since zinc can precipitate in the presence of certain salts, especially phosphates, carbonates and/or sulfates. Its incorporation into hydrogels therefore remains difficult.

Thus, a need remains for the provision of a method making it possible to prepare hydrogels comprising a cross-linked and/or non-cross-linked polysaccharide and also comprising zinc, in particular at concentrations where zinc is biologically active, without precipitation thereof. Advantageously, the proposed method will be as respectful as possible of the properties of hydrogels, i.e., that it will lead to the least possible degradation of the rheological properties of hydrogels during heat sterilization.

### BRIEF DESCRIPTION OF THE INVENTION

The present invention relates to a method for preparing a sterile hydrogel comprising a cross-linked polysaccharide, a non-cross-linked polysaccharide, or a mixture thereof, and further comprising zinc ions, the method comprising the following steps:
(1) preparing a hydrogel comprising a cross-linked polysaccharide, a non-cross-linked polysaccharide, or a mixture thereof, the hydrogel preparation comprising the following steps:
contacting the cross-linked polysaccharide, non-cross-linked polysaccharide or mixture thereof with a physiological saline solution, preferably buffered, the physiological saline solution, preferably buffered, comprising phosphate or carbonate or sulfate salts or mixtures thereof,
adding citrate ions to the cross-linked polysaccharide, non-cross-linked polysaccharide or mixture thereof in a quantity sufficient to obtain a citrate ion concentration of at least 0.1 mM in the hydrogel,
adding zinc ions to the cross-linked polysaccharide, non-cross-linked polysaccharide or mixture thereof in a quantity sufficient to obtain a zinc ion concentration of at most 20 mM in the hydrogel,
   the addition of citrate ions and zinc ions being carried out in a molar ratio of [citrate ions]/[zinc ions] ranging from 1 to 20, and
   provided that the addition of zinc ions is not carried out before the addition of citrate ions when contact with the physiological saline solution, preferably buffered, is carried out before the addition of citrate ions;
(2) sterilizing, preferably by heat, the hydrogel obtained at the end of step (1) to obtain a sterile hydrogel comprising a cross-linked polysaccharide, a non-cross-linked polysaccharide or a mixture thereof and further comprising zinc ions.

The invention also relates to a method for preparing a sterile hydrogel comprising a cross-linked polysaccharide and, optionally, a non-cross-linked polysaccharide, and further comprising zinc ions, the method comprising the following steps:
(0) preparing a cross-linked polysaccharide from a cross-linking reaction medium comprising one or more polysaccharides, one or more cross-linking agents, a solvent and zinc ions in a quantity permitting the preparation of a hydrogel comprising at most 20 mM zinc ions;
(1) preparing a hydrogel from the cross-linked polysaccharide obtained at the end of step (0) and optionally from a non-cross-linked polysaccharide, the preparation of the hydrogel comprising a step of contacting the cross-linked polysaccharide with a physiological saline solution, preferably buffered, comprising phosphate or carbonate or sulfate salts or mixtures thereof;
(2) sterilizing, preferably by heat, the hydrogel obtained at the end of step (1) to obtain a sterile hydrogel;
   wherein:
the cross-linking reaction medium further comprises citrate ions in a quantity sufficient to obtain a concentration of citrate ions of at least 0.1 mM in the hydrogel, the molar ratio of [citrate ions present in the reaction medium]/[zinc ions present in the reaction medium] ranging from 1 to 20; or
step (1) further comprises, before the step of contacting the cross-linked polysaccharide with the physiological saline solution, preferably buffered, a step of adding citrate ions in a quantity sufficient to obtain a concentration of citrate ions of at least 0.1 mM in the hydrogel, the molar ratio of [added citrate ions]/[zinc ions present in the reaction medium] ranging from 1 to 20; or
the physiological saline solution, preferably buffered] further comprises citrate ions in a quantity sufficient to obtain a concentration of citrate ions of at least 0.1 mM in the hydrogel, the molar ratio of [citrate ions present in the physiological saline solution, preferably buffered]/[zinc ions present in the reaction medium] ranging from 1 to 20.

The invention also relates to a method for preparing a sterile hydrogel comprising a cross-linked polysaccharide and, optionally, a non-cross-linked polysaccharide, and further comprising zinc ions, the method comprising the following steps:
(0′) preparing a cross-linking reaction medium comprising:
   one or more polysaccharides,
   one or more cross-linking agents,
   citrate ions in a quantity sufficient to obtain a citrate ion concentration of at least 0.1 mM in the hydrogel,
   zinc ions in a quantity allowing the preparation of a hydrogel comprising at most 20 mM zinc ions, and
   a physiological saline solution, preferably buffered, comprising phosphate or carbonate or sulfate salts or mixtures thereof;
the molar ratio of [citrate ions present in the reaction medium]/[zinc ions present in the reaction medium] ranging from 1 to 20,
the reaction medium being prepared by addition of citrate ions before any contact of zinc ions with the physiological saline solution;
(0) preparing a cross-linked polysaccharide from the reaction medium obtained at the end of step (0′);
(1) preparing a hydrogel from the cross-linked polysaccharide obtained at the end of step (0) and optionally from a non-cross-linked polysaccharide;
(2) sterilizing, preferably by heat, the hydrogel obtained at the end of step (1) to obtain a sterile hydrogel.

The invention also relates to a sterile hydrogel comprising a cross-linked polysaccharide, a non-cross-linked polysaccharide or a mixture thereof, in particular comprising a cross-linked hyaluronic acid, a non-cross-linked hyaluronic acid or a mixture thereof and further comprising zinc and citrate ions obtained by the methods according to the invention.

The invention also relates to the use of citrate ions to protect a hydrogel comprising a cross-linked polysaccharide, a non-cross-linked polysaccharide or mixture thereof, optionally an anesthetic agent, and additionally zinc ions, from degradation of its rheological properties during sterilization, preferably by heat, or to preserve the stability over time of the rheological properties of a hydrogel comprising a cross-linked polysaccharide, a non-cross-linked polysaccharide, or a mixture thereof, and optionally an anesthetic agent and, additionally, zinc ions.

Finally, the invention relates to the use of a solution comprising zinc ions and citrate ions to protect a hydrogel comprising a cross-linked and/or non-cross-linked polysaccharide and, optionally, an anesthetic agent, from degradation of its rheological properties during sterilization, preferably by heat, or to preserve the stability over time of a hydrogel comprising a cross-linked polysaccharide, a non-cross-linked polysaccharide or a mixture thereof and, optionally, an anesthetic agent.

Other aspects of the invention are as described below and in the claims.

### FIGURES

FIG. 1: hydrogel obtained according to the method of the invention (microscope: Olympus SZX16, software: OLYMPUS Stream Start)

FIG. 2: non-invention hydrogel (microscope: Olympus SZX16, software: OLYMPUS Stream Start)

### DETAILED DESCRIPTION OF THE INVENTION

### Definitions

The term “gel” designates a network of polymers which is expanded throughout its volume by a fluid. This means that a gel consists of two media, one solid and the other liquid, dispersed in one another. The so-called solid medium is composed of long polymer molecules connected together by weak bonds (for example hydrogen bonds) or by covalent bonds (cross-linking). The liquid medium is composed of a solvent. A gel generally corresponds to a viscoelastic product which has a phase angle δ of less than or equal to 90°, preferably less than or equal to 70°, preferably less than or equal to 45°, at 1 Hz for a deformation of 0.1% or a pressure of 1 Pa, advantageously a phase angle δ ranging from 2° to 45° or ranging from 20° to 45°.

The term “hydrogel” designates a gel as defined above in which the solvent constituting the liquid medium is predominantly water (for example at least 90%, in particular at least 95%, especially at least 97%, especially at least 98% by weight of the liquid medium) and has a pH ranging from 6.8 to 7.8.

The term “injectable hydrogel” designates a hydrogel that can flow and be injected manually by means of a syringe equipped with a needle of diameter ranging from 0.1 to 0.5 mm, for example a hypodermic needle of 32 G, 30 G, 27 G, 26 G or 25 G.

Preferentially, an injectable hydrogel is a hydrogel having an average extrusion force of less than or equal to 25 N, preferably ranging from 5 to 25 N, more preferably ranging from 8 to 15 N, when measured with a dynamometer, at a fixed speed of approximately 12.5 mm/min, in syringes of external diameter greater than or equal to 6.3 mm, with a needle of external diameter less than or equal to 0.4 mm (27 G) and length of % inch, at room temperature.

A “superficial application” designates the administration, for example by mesotherapy, of a composition superficially into the skin, or onto the skin, for the treatment of the superficial layers of the skin, the epidermis and the most superficial parts of the dermis, to reduce superficial wrinkles and/or to improve the quality of the skin (such as its radiance, density or structure) and/or to rejuvenate the skin.

A “middle application” designates the administration of a composition to the middle part of the skin to treat the middle layers of the skin, as well as to reduce middle wrinkles.

A “deep application” designates the administration of a composition into the deepest layers of the skin, the hypodermis and the deepest part of the dermis, and/or under the skin (above the periosteum) to “add volume”, such as for filling the deepest wrinkles and/or partially atrophied areas of the contour of the face and/or body. The so-called volumizing hydrogels can typically be administered for deep application.

A “cross-linked polysaccharide” designates a polysaccharide modified during a cross-linking reaction.

On the contrary, a “non-cross-linked polysaccharide” designates a polysaccharide not modified with a cross-linking agent and which has therefore not undergone a cross-linking reaction.

The term “cross-linking agent” designates any compound capable of introducing cross-linking between different polysaccharide chains.

The “molar cross-linking rate” (CR), expressed in %, designates the molar ratio of the quantity of cross-linking agent relative to the quantity of repeating unit of the polysaccharide introduced into the cross-linking reaction medium expressed per 100 moles of repeating units of the polysaccharide in the cross-linking medium. For example, a molar cross-linking rate of 1% means that there is one molecule of cross-linking agent introduced into the reaction medium per 100 moles of polysaccharide repeating units.

The expression “repeating unit” of a polysaccharide designates a structural unit composed of one or more (usually 1 or 2) monosaccharides whose repetition produces the complete polysaccharide chain.

The “degree of modification” (MOD) of a polysaccharide, such as hyaluronic acid, is the molar quantity of cross-linking agent bound to the polysaccharide by one or more of its ends, expressed per 100 moles of repeating units of the polysaccharide. It can be determined by methods known to the person skilled in the art, such as nuclear magnetic resonance spectroscopy (NMR). For example, a degree of modification of 1% means that there is one molecule of cross-linking agent per 100 moles of polysaccharide repeating units.

The term “polysaccharide” designates a polymer composed of monosaccharides (preferentially D enantiomers) joined together by glycosidic bonds.

The term “room temperature” is understood to mean a temperature ranging from 20 to 25° C., more particularly 21° C.

The linear viscoelastic region (LVER) corresponds to the range of deformations of the hydrogel from an initial value of elastic modulus G′ to the value of elastic modulus G′ minus 10% of its initial value. The LVER measurement consists of an oscillatory strain sweep measurement in compression mode at a given oscillation frequency to determine the linear viscoelastic region.

### METHODS

The inventors have developed three alternative methods meeting the needs expressed. According to a first variant (method 1), the preparation of a hydrogel comprising a cross-linked and/or non-cross-linked polysaccharide and further comprising zinc is made possible by the addition of citrate ions during the hydrogel preparation phase.

According to a second variant (method 2), when the hydrogel comprises a cross-linked polysaccharide, the preparation of a hydrogel comprising zinc is made possible by cross-linking the polysaccharide in a reaction medium comprising zinc ions and by adding citrate ions either during the cross-linking step or subsequently but before any contact of the zinc ions with a physiological saline solution, preferably buffered, comprising phosphate or carbonate or sulfate salts, alone or mixtures thereof.

According to a third variant (method 3), when the hydrogel comprises a cross-linked polysaccharide, the preparation of a hydrogel comprising zinc is made possible by cross-linking the polysaccharide in a reaction medium comprising zinc ions, citrate ions and a physiological saline solution, preferably buffered, comprising phosphate or carbonate or sulfate salts, alone or mixtures thereof, provided that the citrate ions are added before any contact of the zinc ions with the physiological saline solution.

It has been observed that the addition of citrate ions, in particular citric acid or sodium citrate, or calcium citrate, or potassium citrate or magnesium citrate, to zinc makes it possible to prevent zinc precipitation, especially in the presence of a physiological saline solution, preferably buffered, comprising phosphate or carbonate or sulfate salts or mixtures thereof.

Unexpectedly, it has been observed that the proposed method (variants 1, 2 and 3) also effectively protects the hydrogel from degradation of its rheological properties during sterilization, in particular during heat sterilization. The hydrogels obtained by the method of the present invention thus exhibit fewer changes in their rheological properties after sterilization (better preservation of the elastic modulus G′, better preservation of the phase angle) compared with hydrogels prepared by an equivalent process without addition of zinc and citrate ions, in particular without addition of zinc and citrate ions in the form of a physiological saline solution, preferably buffered, comprising phosphate or carbonate or sulfate salts or mixtures thereof.

Unexpectedly, this protective effect is prolonged over time. The hydrogels obtained by the method of the present invention exhibit better stability over time, i.e., they maintain their rheological properties more effectively over time, especially after sterilization,) than hydrogels prepared by an equivalent process without addition of zinc and citrate ions, in particular without addition of zinc and citrate ions in the form of a physiological saline solution, preferably buffered, comprising phosphate or carbonate or sulfate salts or mixtures thereof, and zinc ions and citrate ions. Indeed, the hydrogels according to the invention exhibit better preservation over time of their rheological properties, especially after sterilization, and do not exhibit precipitation of zinc.

Moreover, it is known that the additional presence of anesthetic in a hydrogel during sterilization, preferably by heat, leads to degradation of the rheological properties of hydrogels based on cross-linked and/or non-cross-linked polysaccharide, in particular hydrogels based on cross-linked and/or non-cross-linked hyaluronic acid. The addition of citrate and zinc ions limits these effects. The hydrogels obtained by the method of the present invention comprising an anesthetic agent exhibit lesser degradation of their rheological properties after sterilization compared to hydrogels comprising an anesthetic agent, prepared by an equivalent process without addition of zinc and citrate ions, in particular without addition of zinc and citrate ions in the form of a physiological saline solution, preferably buffered, comprising phosphate or carbonate or sulfate salts or mixtures thereof.

### Method 1

The present invention thus relates to a method for preparing a sterile hydrogel comprising a cross-linked and/or non-cross-linked polysaccharide, and further comprising zinc ions, the method comprising the following steps:
(1) preparing a hydrogel comprising a cross-linked and/or non-cross-linked polysaccharide, the hydrogel preparation comprising the following steps:
   contacting a cross-linked and/or non-cross-linked polysaccharide with a physiological saline solution, preferably buffered, the saline solution, preferably buffered, comprising phosphate or carbonate or sulfate salts or mixtures thereof;
   adding zinc ions and citrate ions to the cross-linked and/or non-cross-linked polysaccharide in a molar ratio of citrate ions to zinc ions ranging from 1 to 20 and in a quantity sufficient to obtain a concentration of citrate ions of at least 0.1 mM in the hydrogel and a concentration of zinc ions of at most 20 mM in the hydrogel,
      provided that the addition of zinc ions is not carried out before the addition of citrate ions when contacted with the physiological saline solution before the addition of citrate ions;
(2) sterilizing, preferably by heat, the hydrogel obtained at the end of step (1) to obtain a sterile hydrogel.

It is understood that the preparation of the hydrogel from the cross-linked and/or non-cross-linked polysaccharide (step (1)) comprises at least the steps indicated above, the order of these steps being immaterial. Other steps can be implemented.

The Cross-Linked and/or Non-Cross-Linked Polysaccharide

The polysaccharide can be any polymer composed of monosaccharides joined together by glycosidic bonds or mixtures thereof. Preferably, the polysaccharide is chosen from pectin and pectic substances; chitosan; chitin; cellulose and its derivatives; agarose; glycosaminoglycans such as hyaluronic acid, heparosan, dermatan sulfate, keratan sulfate, chondroitin and chondroitin sulfate; and mixtures thereof. Still more preferably, the polysaccharide is chosen from hyaluronic acid, heparosan, chondroitin and mixtures thereof, still more preferentially, the polysaccharide is hyaluronic acid or one of its salts, in particular in a physiologically acceptable salt such as sodium salt, potassium salt, zinc salt, calcium salt, magnesium salt, silver salt, calcium salt and mixtures thereof. More particularly, hyaluronic acid is in acid form or in sodium salt form (NaHA). The hydrogel can thus be a hydrogel based on hyaluronic acid and/or one of its salts.

Preferably, if the polysaccharide is hyaluronic acid or one of its salts, it has a weight average molecular weight (Mw) ranging from 0.05 to 10 MDa, preferentially ranging from 0.5 to 5 MDa, for example ranging from 2 to 4 MDa or ranging from 1 to 5 MDa.

The polysaccharide may be provided in hydrated form (totally or partially hydrated) or in dry form, such as in powder or fiber form. When the polysaccharide is supplied in hydrated form, it is typically in gel form.

A cross-linked polysaccharide can be prepared by any method known to the person skilled in the art.

The cross-linked polysaccharide may result from the reaction of the polysaccharide with a cross-linking agent or from the reaction of a modified polysaccharide to allow the formation of covalent intermolecular bonds.

For example, the cross-linked polysaccharide may be prepared as described in WO 2010131175A1 or WO 201277054A1.

The method of the present invention may thus comprise, before the hydrogel preparation step, a step of preparing a cross-linked polysaccharide.

The cross-linked polysaccharide is preferably a cross-linked polysaccharide whose molar degree of cross-linking is less than or equal to 10%. Preferentially, the cross-linked polysaccharide is a cross-linked polysaccharide whose molar degree of cross-linking is greater than 0 and less than or equal to 6%. Even more preferentially, the cross-linked polysaccharide is a cross-linked polysaccharide whose molar degree of cross-linking is greater than 0 and less than or equal to 4%. Even more preferably, the cross-linked polysaccharide is a cross-linked polysaccharide whose molar degree of cross-linking is greater than 0 and less than or equal to 2%, preferably less than or equal to 1%, even more preferentially less than or equal to 0.8%, especially ranging from 0.1% to 0.5% (number of moles of cross-linking agent(s) per 100 moles of repeating unit of the polysaccharide(s)).

The polysaccharide may be cross-linked by reacting a polysaccharide that has been previously modified. The polysaccharide may have been modified by introducing functional groups capable of reacting with each other and forming covalent intermolecular bonds. The polysaccharide may have been modified by grafting with a molecule allowing subsequent cross-linking of the polysaccharide thus modified. For example, the polysaccharide may have been modified by grafting a silylated molecule, amino acid, amino acid derivative or protein.

The polysaccharide may be cross-linked by means of a cross-linking agent. The polysaccharide is preferably cross-linked by means of a cross-linking agent chosen from epoxide or non-epoxide bi- or multi-functional cross-linking agents, i.e., prepared by reaction of the polysaccharide with a cross-linking agent. Among the epoxide agents that may be mentioned are 1,4-butanediol diglycidyl ether (BDDE), 1,2,7,8-diepoxyoctane, 1,2-bis(2,3-epoxypropyl)-2,3-ethane (EGDGE), poly(ethylene glycol)-diglycidyl ether (PEGDE), and mixtures thereof. Among the non-epoxide agents that may be mentioned are endogenous polyamines such as spermine, spermidine and putrescine, aldehydes such as glutaraldehyde, carbodiimides and divinyl sulfone, hydrazide derivatives such as adipic acid dihydrazide, bisalkoxyamines, dithiols such as polyethylene glycol dithiol and mixtures thereof. Among the non-epoxide agents which may be mentioned are amino acids such as cysteine and lysine; peptides or proteins containing amino acids such as cysteine and lysine; poly(dimethyl siloxane); trimetaphosphates, such as, for example, sodium trimetaphosphate, calcium trimetaphosphate or barium trimetaphosphate.

In some embodiments, the cross-linking agent is an epoxide agent, preferably 1,4-butanediol diglycidyl ether (BDDE) or polyethylene glycol diglycidyl ether. Preferentially, the cross-linking agent is 1,4-butanediol diglycidyl ether (BDDE).

In some embodiments, the cross-linking agent is a non-epoxide agent, preferably chosen from endogenous polyamines, aldehydes, carbodiimides, divinyl sulfone, amino acids, peptides and mixtures thereof.

The cross-linked polysaccharide is preferably a cross-linked polysaccharide having a degree of modification (MOD) of less than or equal to 10%, preferably less than or equal to 6%, preferably less than or equal to 4%, preferably less than or equal to 2%, more preferably less than or equal to 1%. Advantageously, the cross-linked polysaccharide is a cross-linked polysaccharide having a degree of modification (MOD) of less than or equal to 1.8%, more preferably less than or equal to 1.5%, preferentially less than or equal to 1.2%, even more preferentially less than 1%.

In particular, the cross-linked polysaccharide can be prepared by a method comprising the following steps:
(a1) preparing a cross-linking reaction medium comprising one or more polysaccharides, one or more cross-linking agents and a solvent; and
(a2) reacting the reaction medium to obtain a cross-linked polysaccharide.

The polysaccharide is as described above. Preferably, the polysaccharide is hyaluronic acid or a salt of hyaluronic acid, preferably a sodium salt.

In step a1), the polysaccharide is provided in dry form such as in powder or fiber form, or in the hydrated form. When the polysaccharide is supplied in hydrated form, it is in the form of a non-cross-linked gel or solution. In particular, when the polysaccharide is in hydrated form, it is an aqueous non-cross-linked gel or aqueous solution.

The cross-linking agent is as described above.

The solvent is typically water or a mixture comprising water and an organic solvent (typically a mixture comprising at least 90% by weight of water, or at least 95% or at least 99% by weight of water relative to the total weight of the solvent). For example, an organic solvent such as an alcohol, particularly ethanol, or DMSO, can be used to solubilize the cross-linking agent, for example when it is poly(dimethylsiloxane) terminated at each end with a diglycidyl ether (CAS number: 130167-23-6), before its addition to the aqueous reaction medium.

The reaction medium may also comprise salts, pH adjusters, for example a Bronsted base, more preferentially a hydroxide salt, such as sodium or potassium hydroxide, additional components as described below and mixtures thereof. The addition of a Bronsted base can especially be necessary when the functional groups of the cross-linking agent have an epoxide group or a vinyl group. In these cases, cross-linking takes place at a pH greater than or equal to 10, more advantageously greater than or equal to 12, which requires the addition of a Bronsted base to the reaction medium, typically at a concentration comprised between 0.10 M and 0.30 M.

The total quantity of cross-linking agent in the reaction medium typically varies from 0.001 to 0.10 moles per 1 mole of polysaccharide repeating unit, preferably from 0.001 to 0.08 moles or from 0.001 to 0.06 moles per 1 mole of polysaccharide repeating unit, preferentially from 0.001 to 0.04 moles per 1 mole of polysaccharide repeating unit, preferably from 0.001 to 0.03 moles per 1 mole of polysaccharide repeating unit, preferably 0.001 to 0.02 moles per 1 mole of polysaccharide repeating unit, more preferably from 0.001 to 0.01 moles per 1 mole of polysaccharide repeating unit, even more preferably from 0.001 to 0.005 moles per 1 mole of polysaccharide repeating unit.

When the polysaccharide is a glycosaminoglycan such as hyaluronic acid, the repeating unit is a disaccharide unit.

The concentration by mass of polysaccharide or polysaccharide salt in the reaction medium advantageously varies from 50 to 300 mg/g of solvent, preferably from 80 to 200 mg/g.
Step (a1) of the method typically comprises a step of homogenizing the reaction medium. Homogenization is generally carried out by three-dimensional stirring, stirring with a mixer, stirring with blades or stirring with a spatula.
Step (a1) is typically carried out at a temperature ranging from 4 to 35° C., preferably ranging from 15° C. to 25° C. Preferably, the duration of step (1) does not exceed 5 hours. It generally varies from 15 minutes to 4 hours, preferably from 30 minutes to 2 hours.
Step (a2) consists of reacting the reaction medium to obtain a cross-linked polysaccharide. Advantageously, step (a2) is carried out directly after step (a1).

This step makes it possible to cross-link the polysaccharide chains with one another. The functional groups of the cross-linking agent react with functional groups present on the polysaccharides to bind the polysaccharide chains with one another and to cross-link them by forming intermolecular bonds. The cross-linking agent can also react with functional groups present on the same polysaccharide molecule so as to form intramolecular bonds. In particular, the functional groups of the cross-linking agent react with the —OH or —COOH or —CHO groups present on polysaccharides such as hyaluronic acid. cross-linked polysaccharides comprising at least one cross-linking link between two polysaccharide chains, said cross-linking link being the residue of the cross-linking agent are thus obtained.

The cross-linking can be carried out in the presence of several cross-linking agents. When cross-linking is carried out in the presence of several cross-linking agents, the cross-linking agents can be added simultaneously or separately over time to the reaction medium. Step (a2) can thus comprise repeated cross-linking steps; advantageously step (a2) comprises a single cross-linking step. The cross-linking is then carried out in the presence of a total quantity of cross-linking agents typically ranging from 0.1 to 10 moles, or from 0.1 to 8 moles, or from 0.1 to 6 moles, or from 0.1 to 4 moles, or from 0.1 to 3 moles, or from 0.1 to 2 moles or from 0.1 to 1 mole or from 0.1 to 0.8 moles or from 0.1 to 0.5 moles of cross-linking agents (or their salts) per 100 moles of repeating unit of the polysaccharide. The cross-linking conditions, in particular the contents of cross-linking agent, duration and temperatures, as well as the weight average molecular weights (Mw) of the polysaccharide used are interdependent.

The lower the content of cross-linking agent, the longer the reaction time must be in order to obtain similar mechanical properties for the resulting cross-linked polysaccharide, and ultimately of the prepared hydrogel. In other words, the lower the molar percentage of cross-linking agent, the fewer reactive functional groups there are in the reaction medium and the lower the probability that 2 groups will meet and react together, the longer the reaction time must be in order to allow the functional groups to react with one another and form cross-linking bonds, and thus ultimately obtain a hydrogel with desirable properties.

In some embodiments, step (a2) can be carried out by placing the reaction medium directly obtained at the end of step (a1) at a temperature of less than or equal to 30° C., preferably less than or equal to 25° C. The temperature is typically greater than 0° C. or greater than 5° C. or even greater than 10° C. Even more preferably, step (a2) can be carried out by placing the reaction medium directly obtained at the end of step (a1) at a temperature equal to room temperature. When step (a2) is carried out at a temperature less than or equal to 30° C. and greater 0° C., the cross-linking duration is at least 1 minute, preferably at least 10 minutes, even more preferably at least 1 hour. Preferably, the duration of cross-linking is at most 5 days.

In some embodiments, step (a2) can be carried out by placing the reaction medium directly obtained at the end of step (a1) at a temperature greater than 30° C., or greater than or equal to 35° C., or greater than or equal to 40° C., or greater than or equal to 45° C., or greater than or equal to 50° C. The temperature is typically less than 60° C. When the temperature is greater than 30° C., the duration of the cross-linking step is at least greater than or equal to 1 minute, preferably at least greater than or equal to 10 minutes, still more preferably at least 1 hour, preferably comprised between 1 hour and 5 hours.

In some embodiments, step (a2) can be carried out by placing the reaction medium directly obtained at the end of step (a1) at a temperature ranging from 0 to 15° C. or from 1 to 10° C. or from 1 to 9° C.

In some embodiments, step (a2) can be carried out by placing the reaction medium obtained at the end of step (a1), at a pressure P less than or equal to atmospheric pressure and at a temperature T greater than the eutectic point temperature of the reaction medium as measured at pressure P and less than the freezing point temperature of the reaction medium as measured at pressure P, preferably for a period of at least 1 hour. Hydrogels based on cross-linked polysaccharide prepared by such a method are highly biocompatible. Indeed, cross-linked polysaccharides can be prepared with smaller quantities of cross-linking agent, for example quantities ranging from 0.001 to 0.02 moles per 1 mole of repeating unit of the polysaccharide.

The freezing point temperature of the reaction medium designates the temperature at which the mixture of the components of the reaction medium, on a macroscopic scale, solidifies, i.e., becomes non-fluid. Below the freezing point, the mixture is in a freezing state which is characterized by the coexistence of components in solid and liquid form. The freezing state is maintained up to the eutectic point temperature of the reaction medium.

The eutectic point temperature of the reaction medium refers to the temperature below which the mixture of the components of the reaction medium changes from a freezing state (coexistence of liquid and solid phases) to a completely solid state, i.e., a state in which all the components of the mixture are in solid form. The freezing point and eutectic point of a mixture depend on the pressure to which the mixture is subjected so the freezing point and eutectic point are measured at the pressure P.

The freezing point and eutectic point can be determined by differential scanning calorimetry. This method makes it possible to determine the phase transitions. To do this, the product to be studied is gradually cooled until its phase transitions are observed. The temperature T is preferably greater than or equal to −55° C. and less than or equal to −5° C., preferably it ranges from −35° C. to −10° C. More preferably still, the temperature T is approximately −20° C.

The pressure P is preferably atmospheric pressure. Atmospheric pressure is the pressure exerted by the air making up the atmosphere on any surface in contact with it. It varies according to altitude. At an altitude of 0 m, the average atmospheric pressure is 101,325 Pa. Preferably, the pressure P is atmospheric pressure and the temperature T is greater than or equal to −55° C. and less than or equal to −5° C.; preferably T varies from −35° C. to −10° C. or is approximately −20° C.

Preferably, during cross-linking step (a2), when the temperature T is greater than or equal to −55° C. and less than or equal to −5° C., the reaction medium obtained at the end of step (1) is placed for a period of at least 1 hour, preferably at least 3 hours, preferably at least 72 hours, preferably at most 27 weeks under these conditions. Preferably, the cross-linking step (a2) is carried out for a period ranging from 2 to 25 weeks, preferably ranging from 2 to 20 weeks or 2 to 17 weeks, even more preferably from 3 to 8 weeks or 4 to 7 weeks and at temperature T and pressure P.

At the end of step (a2), the cross-linked polysaccharide is typically in the gel form. This gel is generally directly used in the remainder of the method of the invention (step (1)).

The cross-linked and/or non-cross-linked polysaccharides described above are useful for implementing the methods of the invention and thus preparing hydrogels comprising a cross-linked and/or non-cross-linked polysaccharide. The cross-linked or non-cross-linked polysaccharide, or mixture thereof, will constitute the polymer network of the hydrogel. The hydrogel comprising a cross-linked or non-cross-linked polysaccharide or a mixture thereof may thus be said to be based on a cross-linked polysaccharide or a non-cross-linked polysaccharide or a mixture thereof. A hydrogel comprising a non-cross-linked polysaccharide as the only polysaccharide is prepared from a non-cross-linked polysaccharide. A hydrogel comprising a cross-linked polysaccharide as the only polysaccharide is prepared from a cross-linked polysaccharide. When the hydrogel comprises a mixture of a cross-linked and non-cross-linked polysaccharide, the hydrogel is prepared from a cross-linked polysaccharide and a non-cross-linked polysaccharide. The non-cross-linked polysaccharide is typically added to the cross-linked polysaccharide during hydrogel preparation.

### Hydrogel Preparation (Step (1))

The method of the present invention according to method 1 comprises the preparation of a hydrogel comprising a cross-linked and/or non-cross-linked polysaccharide.

The hydrogel preparation comprises the following steps:
contacting the cross-linked and/or non-cross-linked polysaccharide with a physiological saline solution, preferably buffered, the saline solution, preferably buffered, comprising phosphate or carbonate or sulfate salts or mixtures thereof;
adding zinc and citrate ions to the cross-linked and/or non-cross-linked polysaccharide in a molar ratio of citrate ions to zinc ions ranging from 1 to 20 and in a quantity sufficient to obtain a concentration of citrate ions of at least 0.1 mM in the hydrogel and a concentration of zinc ions of at most 20 mM in the hydrogel,
   provided that the addition of zinc ions is not carried out before the addition of citrate ions when the cross-linked and/or non-cross-linked polysaccharide is brought into contact with physiological saline, preferably buffered, before the addition of citrate ions.

When the physiological saline solution is buffered, it is buffered with phosphate and/or carbonate and/or sulfate salts and generally has a physiological pH (6.8-7.8). Generally, the buffered physiological saline solution is a buffered physiological saline solution comprising phosphate salts, preferably the buffered physiological saline solution may be a phosphate buffer. The phosphate buffer may be a PBS buffer with a pH around physiological pH (6.8-7.8) (CAS No: 7647-14-5, 7447-40-7). Preferentially, the buffer is a phosphate buffer, particularly a saline solution buffer of NaH₂PO₄/Na₂HPO₄ or of KH₂PO₄/K₂H PO₄.

When preparing a hydrogel, the cross-linked and/or non-cross-linked polysaccharide may be contacted with a saline solution, preferably buffered, comprising phosphate or carbonate or sulfate salts or mixtures thereof in different steps. For example, the cross-linked and/or non-cross-linked polysaccharide may be contacted with the physiological saline solution, preferably buffered, at the time of adjusting the cross-linked and/or non-cross-linked polysaccharide concentration in the prepared hydrogel. This step is commonly referred to as “dilution”. Thus, during the preparation of the hydrogel, a physiological saline solution, preferably buffered, comprising phosphate or carbonate or sulfate salts or mixtures thereof is typically added during dilution. Alternatively or additionally, the cross-linked and/or non-cross-linked polysaccharide may be contacted with the physiological saline solution, preferably buffered, at the time of pH adjustment or at the time of addition of one or more additional components (see below).

Preferably, in the method of the present invention, the contacting with the physiological saline solution, preferably buffered, comprising phosphate or carbonate or sulfate salts or mixtures thereof is done at least during the step of adjusting the concentration of cross-linked and/or non-cross-linked polysaccharide (dilution).

The quantity of citrate ions added makes it possible to obtain a concentration of citrate ions in the hydrogel of at least 0.1 mM and generally of at most 150 mM, 100 mM, 50 mM, 20 mM or 15 mM. Preferably, the quantity of citrate ions added makes it possible to obtain a concentration of citrate ions in the hydrogel varying from 0.1 to 150 mM or from 0.1 to 100 mM or from 0.1 to 50 mM or from 0.1 to 20 mM or from 0.1 to 15 mM or from 0.3 to 15 mM or from 0.5 to 15 mM or from 1 to 10 mM or from 1.5 to 10 mM or from 2 to 10 mM or from 3 to 10 mM or from 3 to 8 mM or even from 5 to 8 mM.

The molar ratio of citrate ions/repeating units of the polysaccharide may vary from 0.001 to 10 or from 0.001 to 5 or from 0.001 to 4.3, preferentially from 0.001 to 4, even more preferentially from 0.001 to 3. The polysaccharide may have been modified by introducing functional groups capable of reacting with each other and forming covalent intermolecular bonds.

For example, the molar ratio of citrate ions to hyaluronic acid disaccharide units varies from 0.001 to 10 or from 0.001 to 4, preferentially from 0.01 to 3, more preferentially from 0.01 to 2, for example from 0.01 to 1.00 or from 0.015 to 0.500.

The quantity of zinc ions added makes it possible to obtain a concentration of zinc ions in the hydrogel not exceeding 20 mM or not exceeding 7 mM or not exceeding 5 mM or not exceeding 3.5 mM or not exceeding 2 mM or not exceeding 1.6 mM or not exceeding 1.15 mM or not exceeding 0.8 mM. The quantity of zinc ions added makes it possible to obtain a concentration of zinc ions in the hydrogel typically ranging from 0.10 to 20 mM or from 0.10 to 7 mM or from 0.10 to 5 mM or from 0.10 to 3.5 mM or from 0.10 to 2 mM or from 0.10 to 1.6 mM, or from 0.10 to 1.15 mM or from 0.10 to 0.8 mM. In some embodiments, the quantity of zinc ions added makes it possible to obtain a concentration of zinc ions in the hydrogel ranging from 0.10 to 1.6 mM, more preferably from 0.10 to 1.15 mM, even more preferably from 0.1 to 0.8 mM, preferentially from 0.3 to 0.8 mM. The molar ratio of citrate to zinc ions added varies from 1 to 20, preferably from 5 to 20 or from 5 to 15 or from 6 to 15 or from 6 to 10.

The citrate and zinc ions can be added before or after the cross-linked and/or non-cross-linked polysaccharide is contacted with the saline physiological solution, preferably buffered, comprising phosphate or carbonate or sulfate salts or mixtures thereof. The citrate and zinc ions can be added sequentially, either before or after contacting the cross-linked and/or non-cross-linked polysaccharide with the physiological saline solution, preferably buffered. For example, citrate ions can be added and then zinc ions, or zinc ions can be added and then citrate ions. However, zinc ions can only be added before citrate ions when the citrate and zinc ions are added before the cross-linked and/or non-cross-linked polysaccharide is contacted with the physiological saline solution, preferably buffered. Alternatively, citrate and zinc ions can be added concomitantly, either before or after the step of contacting the cross-linked and/or non-cross-linked polysaccharide with the physiological saline solution, preferably buffered.

In other embodiments, the addition of citrate and zinc ions is concomitant with contacting the cross-linked and/or non-cross-linked polysaccharide with the physiological saline solution, preferably buffered. In this case, the physiological saline solution, preferably buffered, may comprise phosphate and/or carbonate and/or sulfate salts or mixtures thereof, and may also comprise citrate and zinc ions.

Citrate ions can be added as a powder or as a solution. The solution may be prepared by adding citric acid into water or physiological saline, preferably buffered, for example into a buffered physiological saline solution comprising phosphate or carbonate or sulfate salts or mixtures thereof.

The solution may be prepared by adding sodium citrate, calcium citrate, potassium citrate or magnesium citrate into water or physiological saline, preferably buffered, for example into a buffered physiological saline solution comprising phosphate or carbonate or sulfate salts or mixtures thereof.

Zinc ions may be added as a powder or as a solution. The solution may be prepared by adding zinc acetate and/or zinc chloride and/or zinc sulfate and/or zinc oxide and/or zinc gluconate to water.

Preferably, zinc and citrate ions may be added in the form of a solution comprising zinc and citrate ions. A solution comprising both citrate and zinc ions is then added during the preparation of the hydrogel. The solution may be prepared by adding citric acid into water and then adding zinc salts (e.g., zinc acetate and/or zinc chloride and/or zinc sulfate and/or zinc oxide and/or zinc gluconate), preferably by adding zinc chloride. The solution may be prepared by adding sodium citrate, calcium citrate, potassium citrate or magnesium citrate into water and then adding zinc salts (e.g., zinc acetate and/or zinc chloride and/or zinc sulfate and/or zinc oxide and/or zinc gluconate), preferably by adding zinc chloride. Alternatively, the solution may be prepared by adding citric acid into physiological saline solution, preferably buffered, followed by adding zinc salts (e.g., zinc acetate and/or zinc chloride and/or zinc sulfate and/or zinc oxide and/or zinc gluconate). Alternatively, the solution may be prepared by adding sodium citrate, calcium citrate, potassium citrate or magnesium citrate into a physiological saline solution, preferably buffered, and then adding zinc salts (e.g., zinc acetate and/or zinc chloride and/or zinc sulfate and/or zinc oxide and/or zinc gluconate). Preferably, the zinc salt is zinc chloride. The buffered physiological solution may be, for example, a phosphate buffer. The phosphate buffer may be a PBS buffer with a pH around physiological pH (6.8-7.8) (CAS No: 7647-14-5, 7447-40-7). Preferentially, the buffer is a phosphate buffer, particularly a saline solution buffer of NaH₂PO₄/Na₂HPO₄ or of KH₂PO₄/K₂HPO₄. If necessary, the pH of the solution can be adjusted to a physiological pH (6.8-7.8), for example by adding sodium hydroxide. Thus, in some embodiments, a phosphate buffer solution comprising zinc and citric acid, having a pH varying from 6.8 to 7.8, is added during the preparation of the hydrogel. When sodium citrate, calcium citrate, potassium citrate or magnesium citrate is used, pH adjustment is typically not necessary.

The preparation of a hydrogel from the cross-linked and/or non-cross-linked polysaccharide can be carried out conventionally, with the difference that zinc ions and citrate ions are added during the preparation of the hydrogel. Thus, the preparation of a hydrogel from a cross-linked and/or non-cross-linked polysaccharide may comprise one or more of the following conventional steps:
pH adjustment (1);
Dilution (2);
Purification (3);
Addition of at least one additional component (4);
Extrusion (5).

These steps well known to the skilled person can be as described below.

They may be at least partly concomitant.

The conventional steps can be carried out in the following sequential manner: optional adjustment of the pH (1), then optional dilution (2), then optional purification (3), then optional addition of an additional component (4), then optional extrusion (5). They may also be carried out in a different order. Advantageously, the extrusion step (5) is carried out last, when at least one of the other conventional steps is implemented. It can also be carried out several times and be inserted between the other conventional steps described.

For example, the conventional steps can be carried out sequentially as follows: (1), (2), (3), (4), (5); or (2), (1), (3), (4), (5); or (2) (1), (4), (5); or (2), (4), (5); or (1), (4), (5); or (2), (4), (3), (5); or (2), (4), (1), (5); or (2), (4), (5); or (4), (2), (1); or (4), (1), (2); or (2), (3), (4), (5); or (4), (2), (3), (5); or (2), (4), (1); or (1), (5), (3), (4); or (1), (5), (4); or (2), (4). Steps (2), (3), (4) and (5) may be concomitant. For example, the preparation of the hydrogel may comprise the following sequence: (2) and (4) are carried out concomitantly.

Citrate ions (in powder form or in solution) and zinc ions (in powder form or in solution) can be added at the time of, before or after any of these conventional steps. Typically when citrate ions are added in powder form, the effect of citrate ions on the pH of the hydrogel can be neutralized. The citrate and zinc ions are preferably added in the form of a solution comprising zinc and citrate ions. The solution is as described above.

The citrate ions (in powder form or in solution) and/or zinc ions (in powder form or in solution) are preferably added during step (2) or (4), preferentially during step (4). Steps (2) and (4) can be carried out concomitantly.

In a variant, the citrate ions and/or zinc ions are added before the extrusion step (5) to obtain a homogeneous gel.

When a purification step (3) is implemented, the citrate ions and/or the zinc ions are advantageously added after the purification step (3). The addition of citrate ions and/or zinc ions after the purification step ensures better control of the citrate ion and/or zinc ion concentration in the prepared hydrogel.

Preferably, citrate ions and/or zinc ions are added between purification step (3) and extrusion step (5).

Citrate and zinc ions can be added after dilution step (2) or during dilution step (2), for example, citrate ions can be added into the aqueous dilution solvent.

Preferably, the citrate ions in powder form or in solution (advantageously the solution comprising citrate ions) and/or the zinc ions are added during the dilution step (2) and/or during the step of adding at least one additional component (4), preferably during the step of adding at least one additional component (4). In particular, in some embodiments, the addition of citrate ions and/or zinc ions (advantageously of the solution comprising citrate and zinc ions) is concomitant with the step of adding at least one additional component (4).

Preferably, the citrate ions in powder form or in solution (advantageously the solution comprising citrate ions) and/or the zinc ions are added during the dilution step (2) and/or during the step of adding at least one additional component (4), preferably during the step of adding at least one additional component (4). In particular, in some embodiments, the addition of citrate ions and/or zinc ions (advantageously of the solution comprising citrate and zinc ions) is concomitant with the step of adding at least one additional component (4).

In particular, in some embodiments, the addition of zinc ions and citrate ions, preferably in the form of a solution comprising citrate and zinc ions, is concomitant with the addition of an anesthetic solution.

In particular, in some embodiments, the addition of zinc ions and citrate ions, preferably in the form of a solution comprising citrate and zinc ions, is concomitant with the addition of a lubricating agent.

In some embodiments, the solution comprising added zinc ions and citrate ions may comprise other components, in particular a lubricating agent, such as non-cross-linked hyaluronic acid, non-cross-linked heparosan, or a mixture thereof.

The steps of dilution (2), addition of at least one additional component (4) and addition of citrate and/or zinc ions may be concomitant.

The citrate and/or zinc ions may be added after the pH adjustment step (1). The citrate ions may be added between the pH adjustment step (1) and extrusion step (5) when these two steps are implemented.

### PH Adjustment (1)

The hydrogel preparation method may comprise a step of adjusting the pH of the hydrogel to reach the desired pH (pH 6.8-7.8).

### Dilution (2)

1 The method for preparing the hydrogel may comprise a step of diluting the cross-linked and/or non-cross-linked polysaccharide. The dilution step makes it possible to adjust the cross-linked and/or non-cross-linked polysaccharide concentration in the prepared hydrogel. In particular, an aqueous solvent is added to the cross-linked and/or non-cross-linked polysaccharide, for example, a physiological saline solution, possibly buffered by the presence of salts, such as phosphate, carbonate or sulfate salts or mixtures thereof. More particularly, the aqueous solvent added has a pH around physiological pH (6.8-7.8). The polysaccharide concentration obtained following the dilution step advantageously varies from 1 mg/g to 50 mg/g of hydrogel, more advantageously from 5 mg/g to 35 mg/g of hydrogel, even more advantageously from 10 mg/g to 30 mg/g of hydrogel.

### Purification (3)

The preparation method according to the invention may comprise at least one purification step. The purification step aims to remove any undesirable impurities. These impurities may result from the cross-linking of the polysaccharide, for example from step (a2) described above. Such impurities may comprise, for example, the residual cross-linking agent, in particular of the epoxide type, which would not have reacted. This step can also be used to perform a liquid exchange, for example a buffer exchange.

The purification step can therefore be implemented most particularly when the hydrogel comprises a cross-linked polysaccharide.

Purification can be carried out by dialysis or else by filtration, for example by dynamic cross-flow filtration (DCF).

### Addition of Additional Components (4)

The method for preparing the hydrogel may comprise a step of adding at least one additional component. The additional component may be chosen from anesthetics, antioxidants, lubricants, amino acids, peptides, proteins such as collagen and silk fibroin, vitamins, elements such as silicon (for example via the addition of orthosilicic acid), minerals, nucleic acids, nucleotides or polynucleotides such as PDRN, nucleosides, coenzymes, adrenergic derivatives, sodium dihydrogen phosphate monohydrate and/or dihydrate, sodium chloride and a mixture thereof.

Non-cross-linked polysaccharides, in particular non-cross-linked hyaluronic acid, non-cross-linked heparosan or mixtures thereof can be mentioned as examples of lubricating agents.

Examples of anesthetics include, in a non-limiting manner, ambucaine, amoxecaine, amylocaine, aprindine, aptocaine, articaine, benzocaine, betoxycaine, bupivacaine, butacaine, butamben, butanilicaine, chlorobutanol, chloroprocaine, cinchocaine, clodacaine, cocaine, cryofluorane, cyclomethycaine, dexivacaine, diamocaine, diperodon, dyclonine, etidocaine, euprocin, febuverine, fomocaine, guafecainol, heptacaine, hexylcaine, hydroxyprocaine, hydroxytetracaine, isobutamben, leucinocaine, levobupivacaine, levoxadrol, lidamidine, lidocaine, lotucaine, menglytate, mepivacaine, meprylcaine, myrtecaine, octacaine, octodrine, oxetacaine, oxybuprocaine, parethoxycaine, paridocaine, phenacaine, piperocaine, piridocaine, polidocanol, pramocaine, prilocaine, procaine, propanocaine, propipocaine, propoxycaine, proxymetacaine, pyrrocaine, quatacaine, quinisocaine, risocaine, rodocaine, ropivacaine, tetracaine, tolycaine, trimecaine, and one of their salts, in particular a hydrochloride, or a mixture thereof. Preferably, the hydrogel according to the invention comprises an anesthetic agent as defined above and in particular lidocaine, mepivacaine or one of their salts such as the hydrochloride.

Examples of antioxidants include, in a non-limiting manner, glutathione, reduced glutathione, ellagic acid, spermine, resveratrol, retinol, L-carnitine, polyols, polyphenols, flavanols, theaflavins, catechins, caffeine, ubiquinol, ubiquinone, alpha-lipoic acid and their derivatives, and a mixture thereof.

Examples of amino acids include, in a non-limiting manner, arginine (e.g., L-arginine), isoleucine (e.g., L-isoleucine), leucine (e.g., L-leucine), lysine (e.g., L-lysine or L-lysine monohydrate), glycine, valine (e.g., L-valine), threonine (e.g., L-threonine), proline (e.g., L proline), methionine, histidine, phenylalanine, tryptophan, cysteine, their derivatives (e.g., N-acetylated derivatives such as N-acetyl-L-cysteine) and a mixture thereof.

Examples of vitamins and their salts include, in a non-limiting manner, vitamins E, A, C, B, especially vitamins B6, B8, B4, B5, B9, B7, B12, and better still pyridoxine and its derivatives and/or salts, preferably pyridoxine hydrochloride.

Examples of minerals include, in a non-limiting manner zinc salts (for example, zinc acetate, especially dehydrated), magnesium salts, calcium salts (for example, hydroxyapatite, especially in bead form), potassium salts, manganese salts, sodium salts, copper salts (for example, copper sulfate, especially pentahydrate), optionally in hydrated form, and mixtures thereof.

Examples of nucleic acids include, in a non-limiting manner, adenosine, cytidine, guanosine, thymidine, cytosine, their derivatives and a mixture thereof. Co-enzymes include coenzyme Q10, CoA, NAD, NADP, and mixtures thereof.

Adrenaline derivatives include adrenaline, noradrenaline and a mixture thereof.

### Extrusion (5)

The hydrogel preparation method may comprise one or more extrusion steps. This extrusion step makes it possible to obtain a more homogeneous hydrogel, in particular with an extrusion force that is as constant as possible, i.e. as regular as possible. For example, the extrusion step can be carried out by means of a sieve whose perforations have a diameter comprised between 50 and 2000 μm. The person skilled in the art knows how to select the perforation diameter according to the mechanical properties of the hydrogel sought.

### Hydrogel Sterilization (Step (2))

The method of the present invention comprises a step of sterilizing the prepared hydrogel. Sterilization is preferably carried out by heat, for example in an autoclave. Sterilization is generally carried out by increasing the temperature of the sterilization medium to a temperature called the plateau temperature, which is maintained for a specified period called the plateau time. Sterilization is preferably carried out at a plateau temperature ranging from 121° C. to 135° C., preferably for a plateau time ranging from 1 minute to 20 minutes with F0≥15. The sterilizing value F0 corresponds to the time required, in minutes, at 121° C., to inactivate 90% of the population of microorganisms present in the product to be sterilized. Alternatively, sterilization can be carried out by gamma radiation, UV radiation or by means of ethylene oxide.

The hydrogel obtained at the end of the method typically has a pH ranging from 6.8 to 7.8 (physiological pH).

### Method 2

The present invention also relates to a method for preparing a sterile hydrogel comprising a cross-linked polysaccharide and, optionally, a non-cross-linked polysaccharide, and further comprising zinc ions, the method comprising the following steps:
(0) preparing a cross-linked polysaccharide from a cross-linking reaction medium comprising one or more polysaccharides, one or more cross-linking agents, a solvent and zinc ions in a quantity permitting the preparation of a hydrogel comprising at most 20 mM citrate zinc;
(1) preparing a hydrogel from the cross-linked polysaccharide obtained at the end of step (0) and optionally from a non-cross-linked polysaccharide, the preparation of the hydrogel comprising contacting the cross-linked polysaccharide with a physiological saline solution, preferably buffered, the physiological saline solution, preferably buffered, comprising phosphate or carbonate or sulfate salts or mixtures thereof;
(2) sterilizing, preferably by heat, the hydrogel obtained at the end of step (1) to obtain a sterile hydrogel;
   wherein:
the cross-linking reaction medium further comprises citrate ions in a quantity sufficient to obtain a concentration of citrate ions of at least 0.1 mM in the hydrogel, the molar ratio of [citrate ions present in the reaction medium]/[zinc ions present in the reaction medium] ranging from 1 to 20; or
step (1) also comprises, before contacting the cross-linked polysaccharide with the physiological saline solution, preferably buffered, a step of adding citrate ions in a quantity sufficient to obtain a concentration of citrate ions of at least 0.1 mM in the hydrogel, the molar ratio of [added citrate ions]/[zinc ions present in the reaction medium] ranging from 1 to 20; or
the physiological saline solution, preferably buffered] further comprises citrate ions in a quantity sufficient to obtain a concentration of citrate ions of at least 0.1 mM in the hydrogel, the molar ratio of [citrate ions present in the physiological saline solution]/[zinc ions present in the reaction medium] ranging from 1 to 20.

In particular, the cross-linked polysaccharide can be prepared by a method comprising the following steps:
(a1) preparing a cross-linking reaction medium comprising one or more polysaccharides, one or more cross-linking agents, a solvent, zinc ions in a quantity sufficient to prepare a hydrogel comprising at most 20 mM zinc ions and optionally citrate ions in a quantity sufficient to obtain a citrate ion concentration of at least 0.1 mM in the hydrogel; and
(a2) reacting the reaction medium to obtain a cross-linked polysaccharide.

When the cross-linked polysaccharide is prepared from a polysaccharide modified beforehand by introducing functional groups capable of reacting with one another and forming covalent intermolecular bonds (i.e., in the absence of cross-linking agent), step (0) comprises preparing a cross-linked polysaccharide from a cross-linking reaction medium comprising one or more modified polysaccharides, a solvent and zinc ions in a quantity making it possible to prepare a hydrogel comprising at most 20 mM zinc ions.

In particular, the cross-linked polysaccharide can be prepared by a method comprising the following steps:
(a1) preparing a cross-linking reaction medium comprising one or more modified polysaccharides, a solvent, zinc ions in a quantity sufficient to prepare a hydrogel comprising at most 20 mM zinc ions and optionally citrate ions in a quantity sufficient to obtain a citrate ion concentration of at least 0.1 mM in the hydrogel; and
(a2) reacting the reaction medium to obtain a cross-linked polysaccharide.

Steps (0), (a1) and (a2) of the method according to method 2 are as described above in the “The cross-linked and/or non-cross-linked polysaccharide” section, with the difference that the cross-linking reaction medium also comprises zinc ions and optionally citrate ions. When the citrate ions are not present in the cross-linking reaction medium, they are added before the step of contacting the cross-linked polysaccharide with the physiological saline solution, preferably buffered, or in the physiological saline solution, preferably buffered. The polysaccharide is as described in the section “The cross-linked and/or non-cross-linked polysaccharide”.

The zinc ions are typically present in the reaction medium in a quantity making it possible not to exceed a concentration of zinc ions in the hydrogel of 20 mM, or of 7 mM, or of 5 mM, or of 3.5 mM, or 2 mM or of 1.6 mM or of 1.15 mM or of 0.8 mM. The zinc ions are typically present in the reaction medium in a quantity making it possible to obtain a concentration of zinc ions in the hydrogel varying from 0.10 to 20 mM or from 0.10 to 7 mM or from 0.10 to 5 mM or from 0.10 to 3.5 mM or from 0.10 to 2 mM or from 0.10 to 1.6 mM, or from 0.10 to 1.15 mM or from 0.10 to 0.8 mM. In some embodiments, the zinc ions are present in the reaction medium in a quantity making it possible to obtain a concentration of zinc ions in the hydrogel varying from 0.10 to 1.6 mM, more preferably from 0.10 to 1.15 mM, even more preferably from 0.1 to 0.8 mM, preferentially from 0.3 to 0.8 mM.

When citrate ions are present in the reaction medium, they are present in a quantity making it possible to obtain a concentration of citrate ions in the hydrogel of at least 0.1 mM and generally of at most 150 mM, 100 mM, 50 mM, 20 mM or 15 mM. Preferably, they are present in a quantity making it possible to obtain a concentration of citrate ions in the hydrogel varying from 0.1 to 150 mM or from 0.1 to 100 mM or from 0.1 to 50 mM or from 0.1 to 20 mM or from 0.1 to 15 mM or from 0.3 to 15 mM or from 0.5 to 15 mM or from 1 to 10 mM or from 1.5 to 10 mM or from 2 to 10 mM or from 3 to 10 mM or from 3 to 8 mM or even from 5 to 8 mM. No covalent bonds are formed between the polysaccharide and the citrate ions.

When the citrate ions are not present in the reaction medium, they are added before being contacted with the physiological saline solution, preferably buffered, or in the physiological saline solution, preferably buffered, in a quantity sufficient to reach the concentrations indicated above.

The molar ratio [citrate ions present in the reaction medium]/[zinc ions present in the reaction medium] or [citrate ions added]/[zinc ions present in the reaction medium] or [citrate ions present in the physiological saline solution, preferably buffered]/[zinc ions present in the reaction medium] varies from 1 to 20, preferably from 5 to 20 or from 5 to 15 or from 6 to 15 or from 6 to 10.

The zinc ions present in the reaction medium may result from the addition of zinc salts to the reaction medium such as zinc sulfate, zinc chloride, zinc gluconate, preferentially zinc chloride.

The citrate ions present in the reaction medium may result from the addition of citric acid, in powder form or in the form of an aqueous solution to the reaction medium.

The citrate ions present in the reaction medium may result from the addition of sodium citrate, calcium citrate, potassium citrate or magnesium citrate, in powder form or in the form of an aqueous solution to the reaction medium.

When the citrate ions are not present in the reaction medium, they are added before the step of contacting with the physiological saline solution, preferably buffered, or in the physiological saline solution, preferably buffered. They may be added as a solution. The solution may be prepared by adding citric acid or sodium citrate or calcium citrate or potassium citrate or magnesium citrate to water or physiological saline solution, preferably buffered, for example to saline solution, preferably buffered, comprising phosphate or carbonate or sulfate salts or mixtures thereof. In other embodiments, the physiological saline solution, preferably buffered, added during the preparation of the hydrogel comprises citrate ions.

At the end of step (0) or (a2), the cross-linked polysaccharide is typically in the form of a gel comprising zinc ions and, optionally, citrate ions. This gel is generally directly used in the remainder of the method of the invention (step (1)).

A hydrogel (step (1)) from the cross-linked polysaccharide obtained at the end of step (0) or (a2) can be prepared conventionally. In particular, the preparation of a hydrogel from the cross-linked polysaccharide obtained at the end of step (0) or (a2) typically comprises one or more of the following conventional steps:
pH adjustment (1);
Dilution (2);
Purification (3);
Addition of at least one additional component (4);
Extrusion (5).

These steps, which are well known to the skilled person, may be as described above in relation to method 1. They may be implemented in the sequential ways described above.

Sterilization (step (2)) is as described in relation to step (2) of method 1.

### Method 3

The invention also relates to a method for preparing a sterile hydrogel comprising a cross-linked polysaccharide and, optionally, a non-cross-linked polysaccharide, and further comprising zinc ions, the method comprising the following steps:
(0′) preparing a cross-linking reaction medium comprising:
   one or more polysaccharides,
   one or more cross-linking agents,
   citrate ions in a quantity sufficient to obtain a citrate ion concentration of at least 0.1 mM in the hydrogel,
   zinc ions in a quantity allowing the preparation of a hydrogel comprising at most 20 mM zinc ions, and
   a physiological saline solution, preferably buffered, comprising phosphate or carbonate or sulfate salts or mixtures thereof;
the molar ratio of [citrate ions present in the reaction medium]/[zinc ions present in the reaction medium] ranging from 1 to 20,
the reaction medium being prepared by addition of citrate ions before any contact of zinc ions with the physiological saline solution;
(0) preparing a cross-linked polysaccharide from the reaction medium obtained at the end of step (0′);
(1) preparing a hydrogel from the cross-linked polysaccharide obtained at the end of step (0) and optionally from a non-cross-linked polysaccharide;
(2) sterilizing, preferably by heat, the hydrogel obtained at the end of step (1) to obtain a sterile hydrogel.

When the cross-linked polysaccharide is prepared from a polysaccharide modified beforehand by introducing functional groups capable of reacting with one another and forming covalent intermolecular bonds (i.e., in the absence of cross-linking agent), step (0′) comprises preparing a cross-linking reaction medium comprising:
one or more previously modified polysaccharides,
citrate ions in a quantity sufficient to obtain a citrate ion concentration of at least 0.1 mM in the hydrogel,
zinc ions in a quantity allowing the preparation of a hydrogel comprising at most 20 mM zinc ions, and
a physiological saline solution, preferably buffered, comprising phosphate or carbonate or sulfate salts or mixtures thereof;
the molar ratio of [citrate ions present in the reaction medium]/[zinc ions present in the reaction medium] ranging from 1 to 20,
the preparation of the reaction medium comprising an addition of citrate ions before any contact of zinc ions with the physiological saline solution.

The components of the reaction medium are as described above.

Step (0) of the method according to method 3 is as described above in the section “The cross-linked and/or non-cross-linked polysaccharide”, with the difference that the cross-linking reaction medium comprises zinc ions, citrate ions and a physiological saline solution, preferably buffered, comprising phosphate or carbonate or sulfate salts or mixtures thereof.

A hydrogel (step (1)) from the cross-linked polysaccharide obtained at the end of step (0) can be prepared conventionally. In particular, the preparation of a hydrogel from the cross-linked polysaccharide obtained at the end of step (0) typically comprises one or more of the following conventional steps:
pH adjustment (1);
Dilution (2);
Purification (3);
Addition of at least one additional component (4);
Extrusion (5).

These steps, which are well known to the skilled person, may be as described above in relation to method 1. They may be implemented in the sequential ways described above.

Sterilization (step (2)) is as described in relation to step (2) of method 1.

### Method 1 or 2 or 3: Optional Step

The method of the present invention (method 1 or 2 or 3) may also comprise a step of packaging the hydrogel. The hydrogel is typically packaged in an injection device. The packaging is preferably carried out just before the sterilization step (step (2)). Thus, the sterile hydrogel can be in the form of an injection device pre-filled with the hydrogel, for example a syringe pre-filled with the hydrogel.

### Sterile Hydrogel

The present invention also relates to a sterile hydrogel comprising a cross-linked and/or non-cross-linked polysaccharide obtained or obtainable by the method of the present invention (method 1 or 2 or 3). The sterile hydrogel comprises zinc ions and citrate ions in a molar ratio of citrate ions to zinc ions ranging from 1 to 20, the concentration of citrate ions in the hydrogel being at least 0.1 mM and the concentration of zinc in the hydrogel not exceeding 20 mM.

Preferably, the concentration of zinc ions in the hydrogel varies from 0.10 to 20 mM or from 0.10 to 7 mM or from 0.10 to 5 mM or from 0.10 to 3.5 mM or from 0.10 to 2 mM or from 0.10 to 1.6 mM, or from 0.10 to 1.15 mM or from 0.10 to 0.8 mM. In some embodiments, the concentration of zinc ions in the hydrogel varies from 0.10 to 1.6 mM, more preferably from 0.10 to 1.15 mM, even more preferably from 0.1 to 0.8 mM, preferentially from 0.3 to 0.8 mM.

Preferably, the concentration of citrate ions in the hydrogel varies from 0.1 to 150 mM or from 0.1 to 100 mM or from 0.1 to 50 mM or from 0.1 to 20 mM or from 0.1 to 15 mM or from 0.3 to 15 mM or from 0.5 to 15 mM or from 1 to 10 mM or from 1.5 to 10 mM or from 2 to 10 mM or from 3 to 10 mM or from 3 to 8 mM or even from 5 to 8 mM.

The sterile hydrogel obtained or obtainable by the method of the present invention (method 1 or 2 or 3) has a physiological pH, i.e., ranging from 6.8 to 7.8. The pH of the sterile hydrogel is preferably greater than or equal to 6.9 and less than or equal to 7.4; 7.3; 7.2; 7.1 or 7.

The sterile hydrogel obtained by the method of the present invention (method 1 or 2 or 3) and comprising a cross-linked polysaccharide, advantageously has a phase angle δ of less than or equal to 45° at 1 Hz for a deformation of 0.1% or a pressure of 1 Pa, preferably a phase angle δ ranging from 2° to 45° or ranging from 20° to 45°.

The hydrogel obtained or obtainable by the method of the present invention is preferably an injectable hydrogel, i.e., one which can flow out and be injected manually by means of a syringe equipped with a needle of diameter ranging from 0.1 to 0.5 mm, for example a hypodermic needle of 32 G, 30 G, 27 G, 26 G, 25 G.

The hydrogel obtained or obtainable by the method of the present invention may comprise from 0.1 to 5% by weight, preferably from 1 to 3% by weight, of polysaccharide (total weight of polysaccharide, i.e., total weight of cross-linked and/or non-cross-linked polysaccharide, for example cross-linked and/or non-cross-linked hyaluronic acid), relative to the total weight of the hydrogel. Thus, when the hydrogel comprises, as the only polysaccharide, a non-cross-linked polysaccharide, the hydrogel obtained by the method of the present invention can therefore comprise from 0.1 to 5% by weight, preferably from 1 to 3% by weight, of non-cross-linked polysaccharide (for example of non-cross-linked hyaluronic acid), relative to the total weight of the hydrogel. When the hydrogel comprises, as sole polysaccharide, a cross-linked polysaccharide, the hydrogel obtained by the method of the present invention can therefore comprise from 0.1 to 5% by weight, preferably from 1 to 3% by weight, of cross-linked polysaccharide (for example cross-linked hyaluronic acid), relative to the total weight of the hydrogel. When the hydrogel comprises the mixture of a cross-linked and non-cross-linked polysaccharide, the hydrogel obtained by the method of the present invention can therefore comprise from 0.1 to 5% by weight, preferably from 1 to 3% by weight, of a mixture of non-cross-linked and cross-linked polysaccharide (for example of non-cross-linked and/or cross-linked hyaluronic acid), relative to the total weight of the hydrogel. In particular, the content of polysaccharide (for example, hyaluronic acid) may vary from 0.5 to 40% by weight, preferentially from 1 to 40% by weight, more preferentially from 5 to 30% by weight, relative to the total weight of polysaccharide (for example, hyaluronic acid) present in the hydrogel.

The total polysaccharide concentration obtained by the method of the present invention advantageously varies from 1 mg/g to 50 mg/g of hydrogel, more advantageously from 5 mg/g to 35 mg/g of hydrogel, even more advantageously from 10 mg/g to 30 mg/g of hydrogel. Preferably the polysaccharide is hyaluronic acid, even more preferentially sodium hyaluronate.

The total polysaccharide concentration in the hydrogel obtained by the method of the present invention advantageously varies from 1 mg/g to 50 mg/g of hydrogel, more advantageously from 5 mg/g to 35 mg/g of hydrogel, even more advantageously from 10 mg/g to 30 mg/g of hydrogel. Preferably the polysaccharide is hyaluronic acid, even more preferentially sodium hyaluronate.

When the hydrogel comprises a cross-linked polysaccharide, the cross-linked polysaccharide preferably has a molar cross-linking rate of less than or equal to 10%. Preferentially, the hydrogel comprises a cross-linked polysaccharide whose molar degree of cross-linking is greater than 0 and less than or equal to 6%. Even more preferentially, the hydrogel comprises a cross-linked polysaccharide whose molar degree of cross-linking is greater than 0 and less than or equal to 4%. Even more preferably, the hydrogel comprises a cross-linked polysaccharide whose molar degree of cross-linking is greater than 0 and less than or equal to 2%, preferably less than or equal to 1%, even more preferentially less than or equal to 0.8%, especially ranging from 0.1% to 0.5% (number of moles of cross-linking agent(s) per 100 moles of repeating unit of the polysaccharide(s)).

When the hydrogel comprises a cross-linked polysaccharide, the cross-linked polysaccharide preferably has a degree of modification (MOD) of less than or equal to 10%, preferably less than or equal to 6%, preferably less than or equal to 4%, preferably less than or equal to 2%, more preferably less than or equal to 1%. Advantageously, the cross-linked polysaccharide has a degree of modification (MOD) of less than or equal to 1.8%, more preferably less than or equal to 1.5%, preferentially less than or equal to 1.2%, even more preferentially less than 1%.

In some embodiments, the hydrogel comprises an anesthetic agent. The anesthetic agent may be as described above, in particular the anesthetic agent may be mepivacaine, lidocaine or one of their salts; more particularly in the form of a hydrochloride salt; preferably in quantities ranging from 0.1 to 30 mg/ml, for example from 0.5 to 10 mg/ml or more preferentially from 2 to 6 mg/ml.

Sterile hydrogels prepared according to the method of the invention are most particularly useful for filling and/or replacing tissues, in particular soft tissues, especially by injecting the hydrogel into the tissue. In addition to filling soft tissues, they can deliver biostimulating effects.

In some embodiments, the sterile hydrogel is injected into the subject subcutaneously. Ideally, the hydrogel allows a slow release of zinc ions in the subject after injection. This release of zinc ions must remain well below the toxic dose of zinc ions. For example, for a hydrogel containing zinc ions, the subcutaneous release in the subject after injection should be less than 0.1 mmol/day.

They can be injected using any of the methods known to the person skilled in the art. In particular, they can be administered by means of an injection device suitable for an intra-epidermal and/or intradermal and/or subcutaneous and/or supraperiosteal injection. The injection device may especially be chosen from a syringe, a set of microsyringes, a wire, a laser or hydraulic device, an injection gun, a needleless injection device, or a microneedle roller.

The sterile hydrogels prepared according to the method of the invention are preferably injected subcutaneously.

They may concern deep applications, middle applications and/or superficial applications. They may have therapeutic and/or cosmetic and/or cosmeceutical applications.

In the cosmetic field, hydrogels can be particularly useful for compensating for tissue volume losses due to aging.

They can be used in the prevention and/or cosmetic treatment of an alteration of the surface appearance of the skin. For example, hydrogels can be used in the cosmetic field for preventing and/or treating the alteration of viscoelastic or biomechanical properties of the skin; for filling in volume defects of the skin, especially for filling wrinkles, fine lines and scars; to reduce nasolabial folds and frown lines, to increase the volume of the cheekbones, chin or lips, to restore facial volume, especially the cheeks, temples, contour of the face and area around the eyes; or to reduce the appearance of wrinkles and fine lines.

The present invention also relates to the cosmetic use of a hydrogel as described above for filling tissues, in particular soft tissues, in particular for compensating tissue volume losses due to aging.

The examples that follow are given for illustrative purposes but should in no way be considered as limiting the present invention.

### EXAMPLES

### 1. Materials

Non-cross-linked sodium hyaluronate
BDDE (Sigma Aldrich)
Citric acid (Sigma Aldrich) (CAS No: 5949-29-1)
Sodium citrate (Sigma Aldrich) (CAS No: 6132-04-3)
Zinc chloride (Sigma Aldrich) (CAS No: 7646-85-7)
Commercial zinc citrate (Thermo Fisher) (CAS No. 5990-32-9)
0.25 M NaOH
1M HCl
PBS phosphate buffer (BBraun),
Lidocaine hydrochloride
Three-dimensional stirrer
DHR-2 rheometer
Dynamometer and test bench
Paddle mill homogenizer
Sterile polyethylene bag

### 2. Methods

### Measuring the Viscoelastic Properties

The viscoelastic properties of the hydrogels obtained were measured using a rheometer (DHR-2) having a stainless steel cone (1°-40 mm) with cone-plane geometry and an anodized aluminum Peltier plate (42 mm) (air gap 24 μm).

0.5 g of sterilized hydrogel is deposited between the Peltier plate and said cone. Then a strain sweep is carried out at 1 Hz and 25° C. The elastic modulus G′, the viscous modulus G″ and the phase angle δ are reported for a stress of 5 Pa. The measurements are carried out in the LVER linear domain.

The stress at the intersection of G′ and G″, τ, is determined at the intersection of the curves of the G′ and G″ moduli and is expressed in Pascals.

### 3. Examples

### 3.1 Example 1a

A hydrogel of cross-linked hyaluronic acid is prepared from a high molecular weight 4 MDa hyaluronic acid and BDDE in a 0.25 M aqueous sodium hydroxide solution (cross-linking for 72 hours at 21° C.). The cross-linked polysaccharide has a degree of cross-linking of 2%. Phosphate buffer and 1 N HCl solution are then added to the cross-linked polysaccharides until a pH of 7.3±0.5 is obtained. The hydrogel obtained is homogenized using a three-dimensional stirrer. The hydrogel is dialyzed. The hydrogel obtained has a concentration of 15 mg of hyaluronic acid per gram of hydrogel.

To the hydrogels obtained are then added:
a solution of high molecular weight sodium hyaluronate as lubricant (same quantity in the various mixtures);
an aqueous solution of lidocaine hydrochloride to obtain 0.3% by weight of lidocaine hydrochloride relative to the weight of the final hydrogel;
optionally a solution comprising zinc and citrate ions.

The solution comprising zinc and citrate ions is prepared as follows. Citric acid (in powder form) is first dissolved in phosphate buffer and then the zinc chloride is dissolved (ZnCl₂ powder) and finally 5 M NaOH is added to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the zinc and citric acid solution.

The solution comprising zinc and citrate ions is added at the same time as the anesthetic solution after the addition of the high molecular weight sodium hyaluronate solution.

The hydrogels obtained were sieved and then packaged in a syringe.

Finally, the hydrogels obtained were sterilized by autoclave (plateau temperature comprised between 121° C. and 135° C. with F0≥15).

After sterilization, the hydrogels A1-A6 were analyzed. No hydrogel shows precipitation. The elastic modulus G′ and the phase angle δ were determined. The results are presented in Table 1 below.

The hydrogels have a molar cross-linking rate of 2%.

TABLE 1

| Hydrogels | A1 | A2 | A3 | A4 | A5 | A6 |
|---|---|---|---|---|---|---|
| Molar concentration of | 0.00 | 0.77 | 2.30 | 3.83 | 5.74 | 7.65 |
| citrate ions (mM) in the | | | | | | |
| final hydrogel | | | | | | |
| Mass percentage of citrate | 0.00 | 0.01 | 0.04 | 0.07 | 0.11 | 0.14 |
| ions in the final hydrogel | | | | | | |
| (% w/w)* | | | | | | |
| Molar zinc concentration in | 0.00 | 0.15 | 0.46 | 0.77 | 1.15 | 1.53 |
| final hydrogel (mM) | | | | | | |
| Mass percentage of zinc in | 0.00 | 0.0010 | 0.0030 | 0.0050 | 0.0075 | 0.0100 |
| the final hydrogel (% w/w)* | | | | | | |
| G′ (1 Hz) before sterilization | 127.7 | 125.8 | 129.1 | 125.5 | 124.6 | 123.3 |
| δ before sterilization | 14.0 | 14.8 | 13.6 | 14.9 | 15.3 | 15.5 |
| ΔG′ (%)¹ | −40.0 | −36.5 | −29.2 | −22.5 | −23.2 | −18.2 |
| Δ δ (%)² | 50.3 | 42.2 | 34.8 | 27.0 | 26 | 22.2 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogels prepared from a method according to the invention comprising a step of adding a solution comprising zinc ions and citrate ions exhibit fewer changes in their rheological properties after sterilization compared to hydrogels prepared by an equivalent method without addition of such a solution.

### 3.2 Example 1b

A hydrogel of cross-linked hyaluronic acid is prepared from a high molecular weight 4 MDa hyaluronic acid and BDDE in a 0.25 M aqueous sodium hydroxide solution (cross-linking for 72 hours at 21° C.). The cross-linked polysaccharide has a degree of modification of 2%. PBS phosphate buffer and 1 N HCl solution are then added to the cross-linked polysaccharides until a pH of 7.3±0.5 is obtained. The hydrogel obtained is homogenized using a three-dimensional stirrer. The hydrogel is dialyzed. The hydrogel obtained has a concentration of 15 mg of hyaluronic acid per gram of product.

To the hydrogels obtained are then added:
a solution of high molecular weight non-cross-linked sodium hyaluronate as lubricant (same quantity in the various mixtures);
optionally a solution comprising zinc and citrate ions.

The solution comprising zinc and citrate ions is prepared as follows. Citric acid (in powder form) is first dissolved in phosphate buffer and then the zinc chloride is dissolved (ZnCl₂ powder) and finally 5 M NaOH is added to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the zinc and citric acid solution.

The solution comprising zinc and citrate ions is added after the addition of the high molecular weight sodium hyaluronate solution.

The hydrogels obtained were sieved and then packaged in a syringe.

Finally, the hydrogels obtained are sterilized by autoclave (plateau temperature comprised between 121° C. and 135° C. with F0≥15).

After sterilization, the hydrogels B1-B4 were analyzed. No hydrogel shows precipitation. The elastic modulus G′, the phase angle δ and the stress at the intersection of G′ and G″, τ were determined. The results are presented in Table 2 below.

The hydrogels have a molar cross-linking rate of 2%.

TABLE 2

| Hydrogels | B1 | B2 | B3 | B4 |
|---|---|---|---|---|
| Molar concentration of citrate | 0.00 | 1.53 | 4.59 | 7.65 |
| ions (mM) in the final hydrogel | | | | |
| Mass percentage of citrate ions | 0.00 | 0.03 | 0.09 | 0.14 |
| in the final hydrogel (% w/w)* | | | | |
| Zinc molar concentration in final | 0.00 | 1.53 | 1.53 | 1.53 |
| hydrogel (mM) | | | | |
| Zinc mass percentage in the final | 0.00 | 0.01 | 0.01 | 0.01 |
| hydrogel)*(% w/w | | | | |
| G′ (1 Hz) before sterilization | 137.8 | 134.7 | 132.5 | 133.7 |
| δ before sterilization | 13.8 | 14.8 | 16.2 | 15.4 |
| ΔG′ (%)¹ | −40.8 | −28.4 | −24.6 | −19.7 |
| Δ δ (%)² | 49.6 | 28.0 | 13.9 | 15.8 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogels prepared from a method according to the invention comprising a step of adding a solution comprising zinc and citrate ions exhibit fewer changes in their rheological properties after sterilization compared to hydrogels prepared by an equivalent method without addition of such a solution.

In addition, it was observed that the addition of a solution comprising zinc and citrate ions preserves the hydrogel structure from degradation over time (Table 3).

TABLE 3

| Hydrogels | B1 | B4 |
|---|---|---|
| G′ (1 Hz) at T0 | 81.5 | 107.3 |
| months | | |
| G′ (1 Hz) T3 months | 71.0 | 106.0 |
| G′ (1 Hz) T6 months | 59.2 | 97.0 |
| ΔG′ (%)¹ at 3 | −12.9 | −1.2 |
| months | | |
| ΔG′ (%)² at 6 | −27.4 | −9.6 |
| months | | |

¹ΔG′ (%) = (G′ T3 months − G′ T0)/(G′ T0) *100
²ΔG′ (%) = (G′ T6 months − G′ T0)/(G′ T0) *100

After 3 months and 6 months at 40° C., the hydrogels prepared from a method according to the invention comprising a step of adding a solution comprising zinc and citrate ions exhibit fewer changes in their rheological properties after sterilization compared to hydrogels prepared by an equivalent method without addition of such a solution. For hydrogel B4 prepared from a method according to the invention comprising a step of adding a solution comprising zinc and citrate ions, no precipitation is observed either after 3 months or after 6 months.

### 3.3 Example 2

For comparison, a hydrogel according to Example 1b is produced with the incorporation of 0.47 g of commercial zinc citrate (in powder form, molar ratio of citrate ions/zinc ions in the hydrogel=0.667) in buffer.

The solution comprising commercial zinc citrate is prepared as follows. The zinc citrate is first dissolved in phosphate buffer and then added to 5 M NaOH to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the solution comprising commercial zinc citrate.

The solution comprising commercial zinc citrate is added after the addition of the high molecular weight sodium hyaluronate solution.

The hydrogels obtained were sieved and then packaged in a syringe.

Finally, the hydrogels obtained were sterilized by autoclave (plateau temperature comprised between 121° C. and 135° C. with F0≥15).

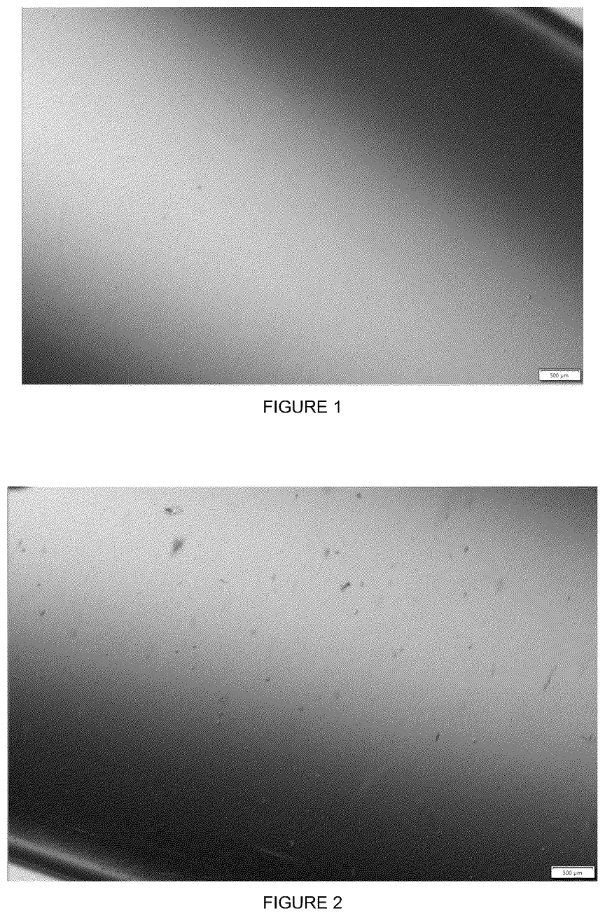

The results of the hydrogels observed under the microscope are presented in FIGS. 1 and 2. The microscope used is: Olympus SZX16, software: OLYMPUS Stream Start.

The hydrogel obtained in accordance with the method according to the invention (FIG. 1—prototype C3) is transparent.

A precipitate is observed as soon as the hydrogel incorporating commercial zinc citrate is sterilized (FIG. 2), which is not desirable for a hydrogel.

### 3.4 Example 3

A study conducted by the NAMSA medical analysis laboratory, according to ISO 10993-23 “Biological Evaluation of Medical Devices, Part 23 (2021): Tests for irritations”, made it possible to evaluate the irritant potential of hydrogels according to the invention after intradermal injection into rabbits.

The control hydrogel C1 and hydrogel according to the invention C2 tested are prepared as follows:

The cross-linked hyaluronic acid hydrogel is prepared from a high molecular weight 1.5 MDa hyaluronic acid and BDDE in a 0.25 M aqueous sodium hydroxide solution (cross-linking for 72 hours at 21° C.). The cross-linked polysaccharide has a degree of modification of 4%. PBS phosphate buffer and 1 N HCl solution are then added to the cross-linked polysaccharides until a pH of 7.3±0.5 is obtained. The hydrogel obtained is homogenized using a three-dimensional stirrer. The hydrogel is then dialyzed. The hydrogels obtained have a concentration of 23 mg of hyaluronic acid per gram of product.

The hydrogel based on a cross-linked polysaccharide obtained is then divided in half.

To the hydrogels obtained are then added:
for hydrogel C1 and C2, an aqueous solution of lidocaine hydrochloride to obtain 0.3% by weight of lidocaine hydrochloride relative to the weight of the final hydrogel and a solution of high molecular weight non-cross-linked sodium hyaluronate as lubricant (same quantity in the various mixtures);
for the hydrogel C2, a solution comprising 0.46 mM of zinc ions and 2.3 mM of citrate ions.

The solution comprising zinc and citrate ions is prepared as follows. Citric acid (in powder form) is first dissolved in phosphate buffer and then the zinc chloride is dissolved (ZnCl₂ powder) and finally 5 M NaOH is added to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the zinc and citric acid solution.

The solution comprising zinc and citrate ions is added after the addition of the high molecular weight sodium hyaluronate solution.

The hydrogels obtained were sieved and then packaged in a syringe.

Finally, the hydrogels obtained are sterilized by autoclave (plateau temperature comprised between 121° C. and 135° C. with F0≥15). No hydrogel shows precipitation.

A 0.2 mL dose of each sterilized hydrogel C1 and C2 is intradermally injected into 5 separate sites on the sides of the back of three rabbits. The injection sites are observed at 24, 48 and 72 h post-injection for signs of erythema and edema, and then daily for up to 28 days. Signs of erythema and edema were measured using a scale of 0-4 for each injection site and for each animal. The overall average score was determined by dividing the sum of the scores by the total number of sites evaluated.

The hydrogel according to the invention C2 has, over the entire duration of the test, a mean irritation score lower than the control hydrogel C1. This thus indicates that the hydrogel C2 prepared according to the invention is less irritating than the control hydrogel.

### 3.5 Example 4

Two hydrogels of cross-linked hyaluronic acid are prepared from a high molecular weight 1.5 MDa hyaluronic acid and BDDE in a 0.25 M aqueous sodium hydroxide solution (cross-linking for 1 month at −20° C.). The cross-linked polysaccharide has a degree of cross-linking of 0.5%. Phosphate buffer and a 1 N HCl solution are then added to the cross-linked polysaccharides until a pH of 7.3±0.5 is obtained. The hydrogel obtained is homogenized using a three-dimensional stirrer. The hydrogel is dialyzed. The hydrogels obtained have a concentration of 23 mg of hyaluronic acid per gram of hydrogel.

To the hydrogels obtained are then added:
a solution of high molecular weight sodium hyaluronate as lubricant (same quantity in the various mixtures);
an aqueous solution of lidocaine hydrochloride to obtain 0.3% by weight of lidocaine hydrochloride relative to the weight of the final hydrogel;
optionally a solution comprising zinc and citrate ions.

The solution comprising zinc and citrate ions is prepared as follows. Citric acid (in powder form) is first dissolved in phosphate buffer and then the zinc chloride is dissolved (ZnCl₂ powder) and finally 5 M NaOH is added to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the zinc and citric acid solution.

The solution comprising zinc and citrate ions is added at the same time as the anesthetic solution after the addition of the high molecular weight sodium hyaluronate solution.

The hydrogels obtained were sieved and then packaged in a syringe.

Finally, the hydrogels obtained were sterilized by autoclave (plateau temperature comprised between 121° C. and 135° C. with F0≥15).

After sterilization, the hydrogels D1 and D2 were analyzed. No hydrogel shows precipitation. The elastic modulus G′ and the phase angle δ were determined. The results are presented in Table 4 below.

The hydrogels have a molar cross-linking rate of 0.5%.

TABLE 4

| Hydrogels | D1 | D2 |
|---|---|---|
| Molar concentration of citrate | 0.00 | 7.0 |
| ions (mM) in the final hydrogel | | |
| Mass percentage of citrate ions | 0.00 | 0.13 |
| in the final hydrogel (% w/w)* | | |
| Zinc molar concentration in final | 0.00 | 0.765 |
| hydrogel (mM) | | |
| Zinc mass percentage in the | 0.00 | 0.005 |
| final hydrogel)(% w/w* | | |
| G′ (1 Hz) before sterilization | 487.5 | 456.2 |
| δ before sterilization | 13.1 | 8.9 |
| ΔG′ (%)¹ | −28.9 | −17.4 |
| Δ δ (%)² | 42.7 | 23.1 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogels prepared from a method according to the invention comprising a step of adding a solution comprising zinc and citrate ions exhibit fewer changes in their rheological properties after sterilization compared to hydrogels prepared by an equivalent method without addition of such a solution.

In addition, it was observed that the addition of a solution comprising zinc and citrate ions preserves the hydrogel structure from degradation over time (Table 5).

TABLE 5

| Hydrogels | D1 | D2 |
|---|---|---|
| G′ (1 Hz) at T0 | 346.8 | 376.7 |
| months | | |
| G′ (1 Hz) T6 months | 238.0 | 340.9 |
| ΔG′ (%)¹ at 6 | −31.4 | −9.5 |
| months | | |

¹ΔG′ (%) = (G′ T6 months − G′ T0)/(G′ T0) *100

After 6 months at 40° C., the hydrogel prepared from a method according to the invention comprising a step of adding a solution comprising zinc and citrate ions (D2) exhibits fewer changes in its rheological properties after sterilization compared to the hydrogel prepared by an equivalent method without addition of such a solution (D1). For hydrogel D2 prepared from a method according to the invention comprising a step of adding a solution comprising zinc and citrate ions, no precipitation is observed after 6 months.

### 3.6 Example 5

Two 20 mg/g non-cross-linked hyaluronic acid hydrogels are prepared from a 1.5 MDa high molecular weight hyaluronic acid in a buffer solution.

The solution comprising zinc added to the hydrogel E1 is prepared as follows. Zinc chloride is dissolved (ZnCl₂ powder) in phosphate buffer and finally 5 M NaOH is added to adjust the pH to a physiological level.

The solution comprising zinc and citrate ions added into the hydrogel E2 is prepared as follows. Citric acid (in powder form) is first dissolved in phosphate buffer and then the zinc chloride is dissolved (ZnCl₂ powder) and finally 5 M NaOH is added to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the zinc and citric acid solution.

Finally, the hydrogels obtained were sterilized by autoclave (plateau temperature comprised between 121° C. and 135° C. with F0≥15).

TABLE 6

| Hydrogels | E1 | E2 |
|---|---|---|
| Molar concentration of citrate ions (mM) in the | 0.00 | 3.0 |
| final hydrogel | | |
| Mass percentage of citrate ions in the final | 0.00 | 0.05 |
| hydrogel (% w/w)* | | |
| Molar concentration of zinc in the final hydrogel | 0.46 | 0.46 |
| (mM) | | |
| Mass percentage of zinc in the final hydrogel | 0.003 | 0.003 |
| (% w/w)* | | |

*1 mL of hydrogel was considered to weigh one gram.

It is observed that the hydrogel prepared from a method according to the invention comprising a step of adding a solution comprising zinc and citrate ions does not exhibit precipitation after sterilization, unlike the hydrogel prepared by an equivalent method without addition of citrate ions.

### 3.7 Example 6

Three hydrogels of cross-linked hyaluronic acid are prepared from a high molecular weight 1.5 MDa hyaluronic acid and BDDE in a 0.25 M aqueous sodium hydroxide solution (cross-linking for 3 hours at 52° C.). The cross-linked polysaccharide has a degree of cross-linking of 8.7%. Phosphate buffer and a 1 N HCl solution are then added to the cross-linked polysaccharides until a pH of 7.3±0.5 is obtained. The hydrogel obtained is homogenized using a three-dimensional stirrer. The hydrogel is dialyzed. The hydrogels obtained have a concentration of 23 mg of hyaluronic acid per gram of hydrogel.

To the hydrogels obtained are then added:
a solution of high molecular weight sodium hyaluronate as lubricant (same quantity in the various mixtures);
an aqueous solution of lidocaine hydrochloride to obtain 0.3% by weight of lidocaine hydrochloride relative to the weight of the final hydrogel;
optionally, a solution comprising zinc and sodium citrate.
optionally, a solution comprising zinc and citric acid.

The solution comprising zinc and citric acid is prepared as follows. Citric acid (in powder form) is first dissolved in phosphate buffer and then the zinc chloride is dissolved (ZnCl₂ powder) and finally 5 M NaOH is added to adjust the pH to a physiological level. The objective is to make a solution that is 100 times more concentrated (in zinc and citrate ions) than the actual concentration desired in the final hydrogel. This avoids an excessive dilution effect of the hydrogel due to the addition of the zinc and citric acid solution.

The solution comprising zinc and sodium citrate is prepared as follows. The sodium citrate (in powder form) is first dissolved in phosphate buffer and then the zinc chloride is dissolved (ZnCl₂ powder).

The solution comprising zinc and citrate ions (from citric acid or sodium citrate) is added at the same time as the anesthetic solution, after the addition of the high molecular weight sodium hyaluronate solution.

The hydrogels obtained were sieved and then packaged in a syringe.

Finally, the hydrogels obtained were sterilized by autoclave (plateau temperature comprised between 121° C. and 135° C. with F0≥15).

After sterilization, the hydrogels F1-F3 were analyzed. No hydrogel shows precipitation. The elastic modulus G′ and the phase angle δ were determined. The results are presented in Table 7 below.

TABLE 7

| | Hydrogels | F1 | F2 | F3 |
|---|---|---|---|---|
| Citric | Molar concentration of | 0.00 | 3.0 | 0.00 |
| acid | citrate ions (mM) in the | | | |
| | final hydrogel | | | |
| | Mass percentage of | 0.00 | 0.06 | 0.00 |
| | citrate ions in the final | | | |
| | hydrogel (% w/w)* | | | |
| Sodium | Molar concentration of | 0.00 | 0.00 | 3.0 |
| citrate | citrate ions (mM) in the | | | |
| | final hydrogel | | | |
| | Mass percentage of | 0.00 | 0.00 | 0.06 |
| | citrate ions in the final | | | |
| | hydrogel (% w/w)* | | | |
| Zinc | Zinc molar concentration | 0.00 | 0.46 | 0.46 |
| | in final hydrogel (mM) | | | |
| | Zinc mass percentage in | 0.00 | 0.003 | 0.003 |
| | the final hydrogel)(% | | | |
| | w/w* | | | |

| | | | |
|---|---|---|---|
| G′ (5 Hz) before sterilization | 166.2 | 163.1 | 167.5 |
| δ before sterilization | 17.1 | 16.9 | 16.4 |
| ΔG′ (%)¹ | −21.5 | −14.6 | −11.9 |
| Δ δ (%)² | 9.7 | 11.0 | 9.5 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogels prepared from a method according to the invention comprising a step of adding a solution comprising zinc and citrate ions exhibit fewer changes in their rheological properties after sterilization compared to hydrogels prepared by an equivalent method without addition of such a solution. In addition, hydrogel F3 comprising a step of adding a solution comprising zinc and citrate ions from sodium citrate shows fewer changes in its rheological properties after sterilization compared to hydrogels prepared by a process comprising zinc and citrate ions from citric acid.

1. A method for preparing a sterile hydrogel comprising a cross-linked polysaccharide, a non-cross-linked polysaccharide, or a mixture thereof, and further comprising zinc ions, the method comprising the following steps:
(1) preparing a hydrogel comprising a cross-linked polysaccharide, a non-cross-linked polysaccharide, or a mixture thereof, the hydrogel preparation comprising the following steps:
contacting the cross-linked polysaccharide, non-cross-linked polysaccharide or mixture thereof with a physiological saline solution, comprising phosphate or carbonate or sulfate salts or mixtures thereof,
adding citrate ions to the cross-linked polysaccharide, non-cross-linked polysaccharide or mixture thereof in a quantity sufficient to obtain a citrate ion concentration of at least 0.1 mM in the hydrogel,
adding zinc ions to the cross-linked polysaccharide, non-cross-linked polysaccharide or mixture thereof in a quantity sufficient to obtain a zinc ion concentration of at most 20 mM in the hydrogel,
the addition of citrate ions and zinc ions being carried out in a molar ratio of [citrate ions]/[zinc ions] ranging from 1 to 20, and
provided that the addition of zinc ions is not carried out before the addition of citrate ions when contact with the physiological saline solution, is carried out before the addition of citrate ions,
(2) sterilizing, the hydrogel obtained at the end of step (1) to obtain a sterile hydrogel comprising a cross-linked polysaccharide, a non-cross-linked polysaccharide or a mixture thereof and further comprising zinc ions.

2. The method according to claim 1 wherein the addition of citrate ions and the addition of zinc ions are carried out after the step of contacting with the physiological saline solution.

3. The method according to claim 1 wherein the addition of citrate ions and the addition of zinc ions are carried out concomitantly, the citrate ions and zinc ions being added as a solution comprising zinc ions and citrate ions.

4. The method according to claim 3 wherein the solution comprising zinc ions and citrate ions is a buffered physiological saline solution.

5. The method according to claim 1 wherein the step (1) of preparing a hydrogel comprises one or more of the following conventional steps:
pH adjustment;
Dilution;
Purification;
Addition of at least one additional component;
Extrusion.

6. A method for preparing a sterile hydrogel comprising a cross-linked polysaccharide, and further comprising zinc ions, the method comprising the following steps:
(0) preparing a cross-linked polysaccharide from a cross-linking reaction medium comprising one or more polysaccharides, one or more cross-linking agents, a solvent and zinc ions in a quantity permitting the preparation of a hydrogel comprising at most 20 mM zinc ions;
(1) preparing a hydrogel from the cross-linked polysaccharide obtained at the end of step (0), the preparation of the hydrogel comprising a step of contacting the cross-linked polysaccharide with a physiological saline solution comprising phosphate or carbonate or sulfate salts or mixtures thereof;
(2) sterilizing, the hydrogel obtained at the end of step (1) to obtain a sterile hydrogel;
wherein:
the cross-linking reaction medium further comprises citrate ions in a quantity sufficient to obtain a concentration of citrate ions of at least 0.1 mM in the hydrogel, the molar ratio of [citrate ions present in the reaction medium]/[zinc ions present in the reaction medium] ranging from 1 to 20; or
step (1) also comprises, before the step of contacting the cross-linked polysaccharide with the physiological saline solution a step of adding citrate ions in a quantity sufficient to obtain a concentration of citrate ions of at least 0.1 mM in the hydrogel, the molar ratio of [added citrate ions]/[zinc ions present in the reaction medium] ranging from 1 to 20; or
the physiological saline solution buffered further comprises citrate ions in a quantity sufficient to obtain a concentration of citrate ions of at least 0.1 mM in the hydrogel, the molar ratio of [citrate ions present in the physiological saline solution]/[zinc ions present in the reaction medium] ranging from 1 to 20.

7. A method for preparing a sterile hydrogel comprising a cross-linked polysaccharide and further comprising zinc ions, the method comprising the following steps:
(0′) preparing a cross-linking reaction medium comprising:
one or more polysaccharides,
one or more cross-linking agents,
citrate ions in a quantity sufficient to obtain a citrate ion concentration of at least 0.1 mM in the hydrogel,
zinc ions in a quantity allowing the preparation of a hydrogel comprising at most 20 mM zinc ions, and
a physiological saline solution comprising phosphate or carbonate or sulfate salts or mixtures thereof;
the molar ratio of [citrate ions present in the reaction medium]/[zinc ions present in the reaction medium] ranging from 1 to 20,
the reaction medium being prepared by addition of citrate ions before any contact of zinc ions with the physiological saline solution;
(0) preparing a cross-linked polysaccharide from the reaction medium obtained at the end of step (0′);
(1) preparing a hydrogel from the cross-linked polysaccharide obtained at the end of step (0) saccharide;
(2) sterilizing the hydrogel obtained at the end of step (1) to obtain a sterile hydrogel.

8. The method according to claim 1 wherein the polysaccharide is a hyaluronic acid.

9. The method according to claim 1 wherein the physiological saline solution is a buffered physiological saline solution comprising phosphate salts.

10. The method according to claim 1, wherein step (1) further comprises a step of adding an anesthetic agent.

11. The method according to claim 1 further comprising a step of packaging the hydrogel after step (1) and before step (2).

12. The method according to claim 1 wherein the sterilization is heat sterilization.

13. A sterile hydrogel comprising a cross-linked polysaccharide, a non-cross-linked polysaccharide or a mixture thereof, and further comprising zinc and citrate ions obtained by the method according to claim 1.

14. The hydrogel according to claim 13 further comprising an anesthetic agent.

15. A method for protecting a hydrogel comprising a cross-linked polysaccharide, a non-cross-linked polysaccharide or a mixture thereof, and additionally zinc ions, from degradation of its rheological properties during sterilization by addition of citrate ions in the hydrogel.

16. A method for preserving the stability over time of the rheological properties of a hydrogel comprising a cross-linked polysaccharide, a non-cross-linked polysaccharide or a mixture thereof, and additionally zinc ions by addition of citrate ions in the hydrogel.

17. A method for protecting a hydrogel comprising a cross-linked and/or non-cross-linked polysaccharide from degradation of its rheological properties during sterilization by addition of a solution comprising citrate ions and zinc ions in the hydrogel.

18. A method for preserving the stability over time of a hydrogel comprising a cross-linked polysaccharide, a non-cross-linked polysaccharide or a mixture thereof by addition of a solution comprising citrate ions and zinc ions in the hydrogel.

19. A method for filling and/or replacing tissues comprising the injection of the sterile hydrogel of claim 13 into the tissue.

20. A cosmetic method for preventing and/or treating the alteration of viscoelastic or biomechanical properties of the skin; for filling in volume defects of the skin; to reduce nasolabial folds and frown lines, to increase the volume of the cheekbones, chin or lips, to restore facial volume; or to reduce the appearance of wrinkles and fine lines comprising administering to a subject the sterile hydrogel of claim 13.
